(19) United States
(12) Patent Application Publication
(10) Pub. No.: US 2011/0229921 A1
(43) Pub. Date: Sep. 22, 2011

(54) METHODS OF ASSAYING URINARY NEUTROPHIL GELATINASE-ASSOCIATED LIPOCALIN (uNGAL) IN THE PROGNOSIS OF CADAVERIC KIDNEY TRANSPLANT FUNCTION IN A PATIENT, INCLUDING A PATIENT DIAGNOSED WITH DELAYED GRAFT FUNCTION (DGF), A METHOD OF ASSAYING uNGAL IN THE ASSESSMENT OF RISK OF DGF IN A PATIENT DIAGNOSED WITH EARLY GRAFT FUNCTION (EGF), AND RELATED KITS

(75) Inventors: Robert C. Doss, Libertyville, IL (US); Sudarshan Hebbar, Chicago, IL (US)

(73) Assignee: ABBOTT LABORATORIES, Abbott Park, IL (US)

(21) Appl. No.: 12/796,794

(22) Filed: Jun. 9, 2010

Related U.S. Application Data
(60) Provisional application No. 61/315,296, filed on Mar. 18, 2010.

(51) Int. Cl. C12Q 1/37

(52) U.S. Cl. 435/23

(57) ABSTRACT

A method of prognosticating cadaveric kidney transplant function in a patient; a method of prognosticating cadaveric kidney transplant function in a patient diagnosed with DGF; a method of assessing risk of DGF in a patient, who has been diagnosed with EGF; and a kit comprising at least one component for assaying urine from a patient for NGAL and instructions for assaying the urine for NGAL and assessing cadaveric kidney transplant function or risk of DGF in the patient.

### CROSS-REFERENCE TO RELATED APPLICATIONS

This application claims the priority of U.S. Provisional Patent Application 61/315,296 filed on Mar. 18, 2010 (pending), incorporated by reference in its entirety.

### TECHNICAL FIELD

The present disclosure relates to methods of prognosticating cadaveric kidney transplant function in a patient, including a patient diagnosed with delayed graft function (DGF), and assessing risk of DGF in a patient diagnosed with early graft function (EGF), by comparing levels of urinary neutrophil gelatinase-associated lipocalin (uNGAL) in the patient at various timepoints, and kits for use in such methods.

### BACKGROUND

Lipocalins are a family of extracellular ligand-binding proteins that are found in a variety of organisms from bacteria to humans. Lipocalins possess many different functions, such as the binding and transport of small hydrophobic molecules, nutrient transport, cell growth regulation, and modulation of the immune response, inflammation and prostaglandin synthesis. Moreover, some lipocalins are also involved in cell regulatory processes and serve as diagnostic and prognostic markers in a variety of disease states. For example, the plasma level of α-glycoprotein is monitored during pregnancy and in the diagnosis and prognosis of conditions such as cancer (e.g., cancer being treated with chemotherapy), renal dysfunction, myocardial infarction, arthritis, and multiple sclerosis.

Neutrophil gelatinase-associated lipocalin (NGAL), which is also known as human neutrophil lipocalin (HNL), N-formyl peptide binding protein, and 25 kDa α2-microglobulin-related protein, among others, is a 24 kDa protein, which can exist as a monomer, a homodimer, or a heterodimer with proteins, such as gelatinase B or matrix metalloproteinase-9 (MMP-9). A trimeric form of NGAL also has been identified. NGAL was originally identified as secreted from specific granules of activated human neutrophils. NGAL now is known to be produced in a variety of epithelial cells, as well as other cell types. Homologous proteins have been identified in mouse (24p3/uterocalin) and rat (α2-microglobulin-related protein/neu-related lipocalin). Structural data have confirmed NGAL has an eight-stranded β-barrel structure, which is characteristic of lipocalins; however, NGAL has an unusually large cavity lined with more polar and positively charged amino acid residues than normally seen in lipocalins. NGAL is believed to bind small lipophilic substances, such as bacteria-derived lipopolysaccharides and formyl peptides, and may function as a modulator of inflammation, among other functions.

NGAL is an early marker for acute kidney injury (AKI) or renal disease (Supavekin et al., Kidney Int. 63: 1714-1724 (2003); Mishra et al., J. Am. Soc. Nephrol. 14: 2534-2543 (2003); Mishra et al., Lancet 365: 1231-1238 (2005); Wagener et al., Anesthesiology 105: 485-491 (2006); and Dent et al., Crit. Care 11: R127 (2007)). In addition to being secreted by specific granules of activated human neutrophils, NGAL is also produced by nephrons in response to tubular epithelial damage and is a marker of tubulointerstitial (TI) injury. NGAL levels rise in acute tubular necrosis (ATN) from ischemia or nephrotoxicity, even after mild “subclinical” renal ischemia. Moreover, NGAL is known to be expressed by the kidney in cases of chronic kidney disease (CKD; see, e.g., Devarajan et al., Amer. J. Kidney Diseases 52(3); 395-399 (September 2008); and Bolignano et al., Amer. J. Kidney Diseases 52(3): 595-605 (September 2008)). Elevated urinary NGAL levels have been suggested to indicate damage in the kidney (Kuwabara et al., Kidney Int. 75: 285-294 (2009)) and to predict progressive kidney failure.

NGAL is secreted into the urine, where it can be easily detected and measured, and precedes the appearance of any other known urinary or serum markers of ischemic injury. The protein is resistant to proteases, suggesting that it can be recovered in the urine as a faithful marker of NGAL expression in kidney tubules. Further, NGAL derived from outside of the kidney, for example, filtered from the blood, does not appear in the urine (Mori et al., J. Clin. Invest. 115: 610-621 (2005)), but rather is quantitatively taken up by the proximal tubule. Therefore, the level of NGAL can be determined in urine and serum (Mishra et al. (2005), supra; and Dent et al. (2007), supra).

AKI occurs in 5% of all hospitalized patients and in up to 50% of patients in intensive care units (ICUs). AKI requiring dialysis is one of the most important independent predictors of death in patients in ICUs. The mortality rate in dialyzed patients in ICUs is about 50-80%, such that about 4 million die each year from AKI. Of those dialyzed ICU patients who survive, about 25% progress to end stage renal/kidney disease (ESRD) within about three years.

Approximately 20,000 kidney transplants are performed annually in the U.S. About 14,000 of those kidney transplants are cadaveric. Kidney injury occurs to some extent in all cadaveric renal allografts, often leading to early renal dysfunction, and increased risk of acute/chronic rejection, chronic allograft nephropathy and graft loss (Perico et al., Lancet 364: 1814-1827 (2004); Ojo et al., Transplantation 63: 968-974 (1997); Koning et al., Transplantation 63: 1620-1628 (1997); Jacobs et al., J. Urol. 171: 47-51 (2004); Lu et al., Kidney Int. 55: 2157-2168 (1999); Sayegh, Kidney Int. 65: 1967-1979 (1999); Boom et al., Kidney Int. 58: 859-866 (2000); Giral-Classe et al., Kidney Int. 54: 972-978 (1998); Shoskes et al., Transplantation 66: 1697-1701 (1998); Troppman et al., Transplant Proc. 31: 1290-1292 (1999); Woo et al., Kidney Int. 55: 692-699 (1999); Tejani et al., Pediatr. Transplant 3: 293-300 (1999); Prommool et al., J. Am. Soc. Nephrol. 11: 565-573 (2000); Halloran et al., Am. J. Transplant 1: 115-120 (2001); and Salahudeen et al., Kidney Int. 65: 713-718 (2004)).

Delayed graft function (DGF), especially when prolonged, complicates the outcome of kidney transplantation (Giral-Classe et al., Kidney Int. 54: 972-978 (1998); and Dominguez et al., Transplant Proc. 41: 131-132 (2009)). DGF is an increasing problem after transplantation of kidneys from deceased donors, given that more kidneys from extended criteria donors (ECD) are being accepted for transplantation.

DGF is usually diagnosed within a few days of transplantation. Diagnosis is based on dieresis, plasma creatinine, and the need for dialysis.

DGF is associated with acute rejection, an increased need for dialysis and post-transplant biopsies, extended post-transplantation hospital stays, and considerable cost (Sola et al., Nephrol. Dial. Transplant 19: iii32-37 (2004); Kyllonen et al., Transpl. Int. 13: 122-128 (2000); Arias, Transplant Proc. 35: 1655-1657 (2003); Giral-Classe et al. (1998), supra; Humar et al., Clin. Transplant 11: 623-627 (1997); Rodrigo et al., Am. J. Transplant 4: 1163-1169 (2004); Almond et al., Transplant Proc. 23: 1304 (1991); and Rosenthal et al., Transplantation 51: 1115-1118 (1991)). The duration of DGF is also associated with a worse one-year outcome (Giral-Classe et al. (1998), supra; and Dominguez et al. (2009), supra). Ischemia-reperfusion injury (IRI) occurs, more or less, in all recipients of kidney transplants from deceased donors, and has a major role in the pathogenesis of DGF. DGF, thus, can be regarded as a form of AKI (Yarlagadda et al., Nephrol. Dial. Transplant 23: 2995-3003 (2008)).

While early forms of AKI are often reversible, the window for early diagnosis and prognosis is narrow, and there are very few early biomarkers. Currently, DGF is untreatable. If DGF could be diagnosed early, timely therapeutic interventions, as used in experimental settings, could become possible in clinical situations. Kidney injury molecule-1 (KIM-1) reportedly is upregulated 24-48 hours after ischemic renal injury; as such, it is a somewhat late marker of tubular cell damage. Cysteine-rich protein 61 (Cyr61) reportedly is detected in urine 3-6 hours after ischemic renal injury in an animal model; however, its detection is cumbersome and lacks specificity. The number of apoptotic tubular cells in donor biopsies either before (Oberbauer et al., J. Am. Soc. Nephrol. 10: 2006-2013 (1999)) or immediately after (Castaneda et al., Transplantation 76: 50-54 (2003)) engraftment has been shown to correlate with early allograft function. Expression of intercellular adhesion molecule 1 (ICAM-1) by immunohistochemistry in donor biopsies obtained prior to engraftment has been shown to predict delayed graft function (Schwarz et al., Transplantation 71: 1666-1670 (2001)).

Urinary NGAL and IL-18 on day zero of cadaveric kidney transplant reportedly predict delayed graft function (DGF) earlier than other methods (Parikh et al., Amer. J. Transplant. 6: 1639-1645 (2006)). NGAL staining intensity in biopsy samples obtained from cadaveric renal allografts within one hour of vascular anastomosis has been shown to predict the degree of AKI (Mishra et al., Pediatr. Nephrol. 21: 856-863 (2006)). A biphasic decline in serum NGAL was reportedly observed in patients receiving kidney transplants from donors after cardiac death (Kusaka et al., Cell Transplant. 17: 129-134 (2008)). Serum NGAL levels reportedly fell significantly as early as one day following kidney transplantation but not in patients with DGF (Lebkowska et al., Transplant Proc. 41: 154-157 (2009)). Urinary NGAL reportedly decreased within 48 hours of kidney transplant in patients with immediate graft function, whereas urinary NGAL reportedly remained overall at about the same level within 48 hours of kidney transplant in patients with slow graft function, and urinary NGAL reportedly increased within 48 hours of kidney transplant in patients with delayed graft function (Hall et al., J. Am. Soc. Nephrol. 1: 189-197 (2010)). NGAL staining intensity reportedly correlates well with peak post-operative serum creatinine as well as the requirement for dialysis 2-3 days later (Mishra et al. (2006), supra). The detection of NGAL in urine following renal tubule cell injury reportedly has been shown to relate to the dose and duration of renal ischemia and nephrotoxemia and to be diagnostic of renal tubule cell injury and renal failure (Devarajan et al., U.S. Pat. App. Pub. No. 2004/0219603).

In view of the foregoing, the present disclosure seeks to provide methods of prognosticating kidney transplant function in a patient, including a patient diagnosed with delayed graft function (DGF). The present disclosure also seeks to provide a method of assessing risk of DGF in a patient, who has been diagnosed with EGF based on (i) the volume of urine voided by the patient within about 24 hours of transplantation being at least about one liter and/or (ii) the level of creatinine in the plasma of the patient at about 24 hours after transplantation being lower than the level of creatinine in the plasma of the patient within about 24 hours before transplantation. In view of the foregoing, the present disclosure also seeks to provide kits for use in such methods. The methods are advantageous inasmuch as they enable earlier prophylactic and/or therapeutic treatment, which, in turn, can increase efficacy and decrease detrimental effects of delayed graft function. Other objects and advantages, as well as inventive features, will become apparent from the detailed description provided herein.

### SUMMARY

A method of prognosticating cadaveric kidney transplant function in a patient is provided. The method comprises assaying urine samples from the patient for urinary neutrophil gelatinase-associated lipocalin (uNGAL), to allow detection of a change in the level of uNGAL in a urine sample from the patient at about 24 hours after cadaveric kidney transplantation in comparison to the level of uNGAL in a urine sample from the patient at within about 24 hours before cadaveric kidney transplantation. According to this method, detecting a decrease in the level of uNGAL of less than or equal to about 55% in a urine sample from the patient at about 24 hours after cadaveric kidney transplantation in comparison to the level of uNGAL in a urine sample from the patient at within about 24 hours before cadaveric kidney transplantation predicts delayed graft function (DGF) and detecting a decrease in the level of uNGAL of at least about 55% in a urine sample from the patient at about 24 hours after cadaveric kidney transplantation in comparison to the level of uNGAL in a urine sample from the patient at within about 24 hours before cadaveric kidney transplantation predicts early graft function (EGF). The method can further comprise determining the volume of urine voided by the patient within about 24 hours of transplantation. A volume of urine that is less than about one liter indicates DGF, whereas a volume of urine that is greater than about one liter indicates EGF.

Another method of prognosticating cadaveric kidney transplant function in a patient is provided. The method comprises assaying urine samples from the patient for uNGAL to allow detection of a change in the level of uNGAL in a urine sample from the patient at about 24 hours after cadaveric kidney transplantation in comparison to the level of uNGAL in a urine sample from the patient at within about 24 hours before cadaveric kidney transplantation. According to this method, detecting a decrease in the level of uNGAL of less than or equal to about 20% in a urine sample from the patient at about 24 hours after cadaveric kidney transplantation in comparison to the level of uNGAL in a urine sample from the patient at within about 24 hours before cadaveric kidney transplantation predicts DGF lasting more than about 14 days. The method can further comprise assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation, wherein detecting an increase in the level of uNGAL in a urine sample from the patient at about 72 hours after cadaveric kidney transplantation in comparison to the level of uNGAL in a urine sample from the patient at about 24 hours after cadaveric kidney transplantation further predicts DGF lasting more than about 14 days.

Yet another method of prognosticating cadaveric kidney transplant function in a patient is provided. The method comprises assaying urine samples from the patient for uNGAL to allow detection of a change in the level of uNGAL in a urine sample from the patient at about 24 hours after cadaveric kidney transplantation in comparison to the level of uNGAL in a urine sample from the patient at within about 24 hours before cadaveric kidney transplantation. Detecting a decrease in the level of uNGAL of at least about 20% and less than about 35% in a urine sample from the patient at about 24 hours after cadaveric kidney transplantation in comparison to the level of uNGAL in a urine sample from the patient at within about 24 hours before cadaveric kidney transplantation predicts DGF lasting less than about 14 days. The method can further comprise assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation. Detecting a decrease in the level of uNGAL of at least about 30% in a urine sample from the patient at about 72 hours after cadaveric kidney transplantation in comparison to the level of uNGAL in a urine sample from the patient at about 24 hours after cadaveric kidney transplantation predicts DGF lasting less than or equal to (“≦”) 7 days, whereas a decrease in the level of uNGAL of less than about 10% in a urine sample from the patient at about 72 hours after cadaveric kidney transplantation in comparison to the level of uNGAL in a urine sample from the patient at about 24 hours after cadaveric kidney transplantation predicts DGF lasting from about 8 days to about 14 days.

Further provided is a method of prognosticating cadaveric kidney transplant function in a patient diagnosed with DGF. The method comprises assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation. According to this method, detecting adecrease in the level of uNGAL of at least about 30% predicts DGF lasting ≦7 days.

Still further provided is another method of prognosticating cadaveric kidney transplant function in a patient diagnosed with DGF. The method comprises assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation. According to this method, detecting a decrease in the level of uNGAL of less than about 10% in a urine sample from the patient at about 72 hours after cadaveric kidney transplantation in comparison to the level of uNGAL in a urine sample from the patient at about 24 hours after cadaveric kidney transplantation predicts DGF lasting from about 8 days to about 14 days.

Even still further provided is another method of prognosticating cadaveric kidney transplant function in a patient diagnosed with DGF. The method comprises assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation. According to this method, detecting an increase in the level of uNGAL in a urine sample from the patient at about 72 hours after cadaveric kidney transplantation in comparison to the level of uNGAL in a urine sample from the patient at about 24 hours after cadaveric kidney transplantation predicts DGF lasting longer than about 14 days.

A method of assessing risk of DGF in a patient, who has been diagnosed with EGF based on (i) the volume of urine voided by the patient within about 24 hours of transplantation being at least about one liter and/or (ii) the level of creatinine in the plasma of the patient at about 24 hours after transplantation being lower than the level of creatinine in the plasma of the patient within about 24 hours before transplantation, is also provided. The method comprises assaying urine samples from the patient for uNGAL to allow detection of a change in the level of uNGAL in a urine sample from the patient at about 24 hours after cadaveric kidney transplantation in comparison to the level of uNGAL in a urine sample from the patient at within about 24 hours before cadaveric kidney transplantation. According to this method, detecting a decrease in the level of uNGAL of less than about 55% in a urine sample from the patient at about 24 hours after cadaveric kidney transplantation in comparison to the level of uNGAL in a urine sample from the patient within about 24 hours before cadaveric kidney transplantation predicts risk of DGF.

In view of the above, provided is a kit comprising at least one component for assaying urine from a patient, who has received a cadaveric kidney transplant or is about to receive a cadaveric kidney transplant, for NGAL and instructions for assaying the urine for NGAL and assessing cadaveric kidney transplant function in the patient.

Also in view of the above, provided is a kit comprising at least one component for assaying urine from a patient, who has received a cadaveric kidney transplant and has been diagnosed with EGF, for NGAL and instructions for assaying the urine for NGAL and assessing risk of DGF in the patient.

### BRIEF DESCRIPTION OF FIGURES

FIG. 1 is a bar graph of time of uNGAL sampling (pre-transplantation (pre-TX), day 1, day 3, day 7 and day 14) vs. concentration of uNGAL in ng/mL. The white bars represent transplant recipients with DGF, whereas the black bars represent transplant recipients with EGF.

FIG. 2 is a bar graph of time of uNGAL sampling (pre-TX, day 1, day 3, day 7 and day 14) vs. concentration of uNGAL in ng/mL in which the white bars represent transplant recipients with DGF for less than or equal to 7 days, the gray bars represent transplant recipients with DGF for 8-14 days, and the black bars represent transplant recipients with DGF for more than 14 days.

FIG. 3a is a graph of specificity vs. sensitivity for uNGAL concentration on day 1 in predicting DGF lasting longer than 7 days.

FIG. 3b is a graph of specificity vs. sensitivity for uNGAL concentration on day 1 in predicting DGF lasting longer than 14 days.

FIG. 3c is a graph of specificity vs. sensitivity for uNGAL concentration on day 3 in predicting DGF lasting longer than 14 days.

### DETAILED DESCRIPTION

The present disclosure is predicated, at least in part, on the discovery that cadaveric kidney transplant function in a patient can be prognosticated by comparing the level of urinary neutrophil gelatinase-associated lipocalin (uNGAL) in the patient at various points in time before and after kidney transplantation. The patient can be one who has been diagnosed with delayed graft function (DGF). The present disclosure is further predicated on the discovery that risk of DGF can be assessed in a patient diagnosed with early graft function (EGF) by comparing the level of uNGAL in the patient at various points in time after kidney transplantation.

### DEFINITIONS

The following terms are relevant to the present disclosure:

(a) “About” refers to approximately a +/−10% variation from the stated value. It is to be understood that such a variation is always included in any given value provided herein, whether or not specific reference is made to it.

(b) “Antibody” and “antibodies” refer to monoclonal antibodies, multispecific antibodies, human antibodies, humanized antibodies (fully or partially humanized), animal antibodies (such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA and IgY), class (for example, IgG₁, IgG₂, IgG₃, IgG₄, IgA₁ and IgA₂) or subclass. An antibody, whose affinity (namely, K_{D}, k_{d} or kₐ) has been increased or improved via the screening of a combinatory antibody library that has been prepared using bio-display, is referred to as an “affinity maturated antibody.” For simplicity sake, an antibody against an analyte is frequently referred to herein as being either an “anti-analyte antibody,” or merely an “analyte antibody” (e.g., an anti-NGAL antibody or an NGAL antibody).

(c) “Antibody fragment” and “antibody fragments” refer to a portion of an intact antibody comprising the antigen-binding site or variable region. The portion does not include the constant heavy chain domains (i.e., CH2, CH₃ or CH₄, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

(d) “Binding Constants” are as described herein. The term “association rate constant,” “kₒₙ,” or “kₐ” as used interchangeably herein, refers to the value indicating the binding rate of an antibody to its target antigen or the rate of complex formation between an antibody and antigen as shown by the equation:

Antibody(“Ab”)+Antigen(“Ag”)→Ab−Ag.

The term “dissociation rate constant,” “k_{off}” or “k_{d}” as used interchangeably herein, refers to the value indicating the dissociation rate of an antibody from its target antigen or separation of Ab−Ag complex over time into free antibody and antigen as shown by the equation: Ab+Ag←Ab−Ag.

Methods for determining association and dissociation rate constants are well known in the art. Using fluorescence-based techniques offers high sensitivity and the ability to examine samples in physiological buffers at equilibrium. Other experimental approaches and instruments, such as a BIAcore® (biomolecular interaction analysis), can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), also can be used.

The term “equilibrium dissociation constant” or “K_{D}” as used interchangeably, herein, refers to the value obtained by dividing the dissociation rate (k_{off}) by the association rate (kₒₙ). The association rate, the dissociation rate and the equilibrium dissociation constant are used to represent the binding affinity of an antibody to an antigen.

(e) “Component,” “components,” and “at least one component,” refer generally to a capture antibody, a detection or conjugate antibody, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a urine sample in accordance with the methods described herein and other methods known in the art. Some components can be in solution or lyophilized for reconstitution for use in an assay.

(f) “Control” refers to a composition known to not contain NGAL (or a fragment thereof) (“negative control”) or to contain NGAL (or a fragment thereof) (“positive control”). A positive control can comprise a known concentration of NGAL (or a fragment thereof). “Control,” “positive control,” and “calibrator” may be used interchangeably herein to refer to a composition comprising a known concentration of NGAL (or a fragment thereof). A “positive control” can be used to establish assay performance characteristics and is a useful indicator of the integrity of reagents (e.g., analytes).

(g) “Delayed graft function” or “DGF” generally is defined as described by Halloran et al. (Transplantation 46: 223-228 (1988)), i.e., oliguria of less than 1 L/24 hours for more than two days, plasma creatinine concentration greater than 500 μmol/l throughout the first week, or more than one dialysis session needed in the first week. Alternately, DGF can be defined using the conventional definition of Humar et al. (Clin. Transplant 11: 623-627 (1997)), i.e., the need for dialysis during the first week after transplantation. “Early graft function” or “EGF” (sometimes known as immediate graft function) generally is considered an absence of DGF, and is assessed, e.g., by urine output during the first 24 hours and serum or plasma creatinine during the first week after transplantation, as well as by dialysis dependency during the first week.

(h) “Epitope,” “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

(i) “Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

(j) “Label” and “detectable label” mean a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

(k) “Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Exemplary linking sequences include, but are not limited to: (i) Histidine (H is) tags, such as a 6×His tag, useful as linking sequences to facilitate the isolation and purification of polypeptides and antibodies of interest; (ii) Enterokinase cleavage sites, like His tags, useful in the isolation and purification of proteins and antibodies of interest. Often, enterokinase cleavage sites are used together with His tags in the isolation and purification of proteins and antibodies of interest. Various enterokinase cleavage sites are known in the art; (iii) Miscellaneous sequences can be used to link or connect the light and/or heavy chain variable regions of single chain variable region fragments. Examples of other linking sequences can be found in Bird et al., Science 242: 423-426 (1988); Huston et al., PNAS USA 85: 5879-5883 (1988); and McCafferty et al., Nature 348: 552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

(l) “Neutrophil gelatinase-associated lipocalin (NGAL),” which is also known as human neutrophil lipocalin (HNL), N-formyl peptide binding protein, and 25 kDa α2-microglobulin-related protein, is a 24 kDa protein, which can exist as a monomer, a homodimer, or a heterodimer with proteins, such as gelatinase B or matrix metalloproteinase-9 (MMP-9). See, e.g., Kjeldsen et al., J. Biol. Chem. 268 (14): 15 10425-10432 (1993), for an exemplary amino acid sequence. While a signal peptide may or may not be present, generally, when present, the signal peptide comprises amino acids 1-20. Therefore, all amino acid sequences are typically numbered from the N-terminus to the C-terminus with the signal peptide present. If the signal peptide is not present, the first amino acid is numbered 21.

The NGAL polynucleotide or polypeptide can be any NGAL sequence, e.g., including that set forth as Genbank accession numbers Genpept CAA58127, AAB26529, XP_{—}862322, XP_{—}548441, P80108, P11672, X83006.1, X99133.1, CAA67574.1, BCO33089.1, AAH33089.1, S75256.1, AD14168.1, JC2339, 1DFVA, 1DFVB, 1L6MA, 1L6 MB, 1L6MC, 1NGLA, 1QQSA, 1X71A, 1X71B, 1X71C, 1X89A, 1X89B, 1X89C, 1X8UA, 1X8UB, and 1X8UC. NGAL polynucleotide and polypeptide (e.g., polyamino acid) sequences are as found in nature, based on sequences found in nature, isolated, synthetic, semi-synthetic, recombinant, or other. In one embodiment, the NGAL is human NGAL (also known as “hNGAL”). NGAL polypeptide sequences can be of the mature human NGAL sequence (sequence not including the 20-residue amino acid signal peptide typically found in nature, and/or minus any other signal peptide sequence). When a signal peptide is present, it is numbered, e.g., as residues 1 to 20, with comparable numbering applied for the encoding polynucleotide sequence.

Likewise, an initial Met residue at the N-terminus of NGAL is present only in NGAL produced in prokaryotes (e.g., E. coli), or in synthetic (including semi-synthetic) or derived sequences, and not in NGAL produced in eukaryotes (e.g., mammalian cells, including human and yeast cells). Consequently, when present, an initial Met residue is typically counted as a negative number, e.g., as residue −1, with no similar numbering adjustment being made for the polynucleotide sequence in a prokaryotic versus eukaryotic background or expression system inasmuch as the polynucleotide sequence is replicated and transcribed the same in both backgrounds, and the difference lies at the level of translation.

Accordingly, the disclosure herein encompasses a multitude of different NGAL polynucleotide and polypeptide sequences as present and/or produced in a prokaryotic and/or eukaryotic background (e.g., with consequent optimization for codon recognition). In sum, the sequences may or may not possess or encode: (a) a signal peptide; (b) an initiator Met residue present in the mature NGAL sequence at the N-terminus; (c) an initiator Met residue present at the start of a signal peptide that precedes the mature NGAL protein; and (d) other variations such as would be apparent to one skilled in the art.

“NGAL fragment” refers to a polypeptide that comprises a part that is less than the entirety of a mature NGAL (e.g., human NGAL) or NGAL including a signal peptide. A fragment of NGAL contains at least one contiguous or nonlinear epitope of NGAL. The precise boundaries of such an epitope can be confirmed using ordinary skill in the art. The epitope can comprise at least about 5 contiguous amino acids, such as about 10 contiguous amino acids, about 15 contiguous amino acids, or about 20 contiguous amino acids.

(m) “NGAL hybrid” or “NGAL hybridoma” refers to a particular hybridoma clone or subclone (as specified) that produces an anti-NGAL antibody of interest. Generally, there may be some small variation in the affinity of antibodies produced by a hybridoma clone as compared to those from a subclone of the same type, e.g., reflecting purity of the clone. By comparison, it is well-established that all hybridoma subclones originating from the same clone and further, that produce the anti-NGAL antibody of interest, produce antibodies of identical sequence and/or identical structure.

(n) “Patient” and “subject” may be used interchangeably herein to refer to an animal, such as a bird (e.g., a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, and a mouse) and a primate (for example, a monkey, a chimpanzee, and a human). Preferably, the patient or subject is a human, such as a non-pregnant human, a pregnant human, a post-partum human, a human at risk for preeclampsia, a human having preeclampsia, a human at risk for cardiovascular disease, or a human having cardiovascular disease.

(o) “Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). The present disclosure provides exemplary predetermined levels, as well as means of determining predetermined levels (e.g., by so-called “ROC” or Receiver Operating Characteristic curve analysis). However, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, the correlations and the methods as described herein should be generally applicable.

(p) “Pretreatment reagent,” e.g., lysis, precipitation and/or solubilization reagent, as used in a diagnostic assay as described herein is one that lyses any cells and/or solubilizes any analyte that is/are present in a urine sample. Pretreatment is not necessary for all samples, as described further herein. Among other things, solubilizing the analyte (i.e., NGAL or NGAL fragment) entails release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the urine sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte.

(q) “Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

(r) “Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (b) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

(s) “Risk” refers to the possibility or probability of a particular event occurring either presently, or, at some point in the future. “Risk stratification” refers to an array of known clinical risk factors that allows physicians to classify patients into a low, moderate, high or highest risk of developing a particular disease, disorder or condition.

(t) “Sample,” “urine sample,” and “patient sample” may be used interchangeably herein. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

(u) “Series of calibrating compositions” refers to a plurality of compositions comprising a known concentration of NGAL (or fragment thereof), wherein each of the compositions differs from the other compositions in the series by the concentration of NGAL (or fragment thereof).

(v) “Solid phase” refers to any material that is insoluble, or can be made insoluble by a subsequent reaction. The solid phase can be chosen for its intrinsic ability to attract and immobilize a capture agent. Alternatively, the solid phase can have affixed thereto a linking agent that has the ability to attract and immobilize the capture agent. The linking agent can, for example, include a charged substance that is oppositely charged with respect to the capture agent itself or to a charged substance conjugated to the capture agent. In general, the linking agent can be any binding partner (preferably specific) that is immobilized on (attached to) the solid phase and that has the ability to immobilize the capture agent through a binding reaction. The linking agent enables the indirect binding of the capture agent to a solid phase material before the performance of the assay or during the performance of the assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

(w) “Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

(x) “Specific” and “specificity” in the context of an interaction between members of a specific binding pair (e.g., an antigen (or a fragment thereof) and an antibody (or antigenically reactive fragment thereof)) refer to the selective reactivity of the interaction. The phrase “specifically binds to” and analogous phrases refer to the ability of antibodies (or antigenically reactive fragments thereof) to bind specifically to an antigen, such as NGAL (or a fragment thereof), and not bind specifically to other antigens (or fragments thereof).

(y) “Substantially identical” as used herein means that a first sequence and a second sequence are at least from about 50% to about 99% identical over a region from about 8 to about 100 or more residues (including, in particular, any range from about 8 to about 100 residues).

(z) “Tracer” means an analyte or analyte fragment conjugated to a label, such as NGAL conjugated to a fluorescein moiety, wherein the analyte conjugated to the label can effectively compete with the analyte for sites on an antibody specific for the analyte.

(aa) “Urine component” and “urine components” refer generally to any biological or chemical component(s) that can occur in urine, including, but not limited to, proteins, nucleic acids, fatty acids, cells, bacteria, viruses, chemical compounds, and drugs.

(bb) “Variant” as used herein means a polypeptide that differs from a given polypeptide (e.g., anti-NGAL antibody) in amino acid sequence by the insertion, deletion, or conservative substitution of amino acids, but that retains the biological activity of the given polypeptide (i.e., can compete with anti-NGAL antibody as defined herein for binding to NGAL). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol. 157: 105-132 (1982)). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101, which is incorporated herein by reference). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-NGAL antibody that differs from the corresponding fragment of anti-NGAL antibody in amino acid sequence but is still antigenically reactive and can compete with the corresponding fragment of anti-NGAL antibody for binding with NGAL. “Variant” also can be used to describe a polypeptide or a fragment thereof that has been differentially processed, such as by proteolysis, phosphorylation, or other post-translational modification, yet retains its antigen reactivity, i.e., ability to bind to NGAL.

The above terminology is provided for the purpose of describing particular embodiments. The terminology is not intended to be limiting.

### Method of Determining the Level of NGAL in a Urine Sample

The present disclosure provides a method for determining the level of NGAL (or a fragment thereof) in a urine sample. Any suitable assay as is known in the art can be used in the method. Examples include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay, a capture reagent that specifically binds NGAL (or a fragment thereof) of interest is attached to the surface of a mass spectrometry probe, such as a pre-activated protein chip array. The NGAL (or a fragment thereof) is then specifically captured on the biochip, and the captured NGAL (or a fragment thereof) is detected by mass spectrometry. Alternatively, the NGAL (or a fragment thereof) can be eluted from the capture reagent and detected by traditional MALDI (matrix-assisted laser desorption/ionization) or by SELDI. A chemiluminescent microparticle immunoassay, in particular one employing the ARCHITECT® automated analyzer (Abbott Laboratories, Abbott Park, Ill.), is an exemplary immunoassay that can be employed as described herein, especially a NGAL Assay for the Abbott ARCHITECT® and marketed by Abbott Laboratories (e.g., Abbott Park, Ill. or Longford, Ireland). Another exemplary assay is described, e.g., in United States Patent Application Nos. US 2009-0123946 and US 2009-0269777 (incorporated by reference for their teachings regarding same). Since a small sample volume is used in the ARCHITECT® automated analyzer, and the assay is based on a urine matrix as opposed to a serum matrix, there should be less interference (e.g., due to the presence of hemoglobuin, bilirubin, HAMA, triglycerides, and the Rh factor).

Methods well-known in the art for collecting, handling and processing urine are used in the practice of the present disclosure, for instance, when the antibodies according to the present disclosure are employed as immunodiagnostic reagents, and/or in a NGAL immunoassay kit. The urine sample can comprise further moieties in addition to the NGAL analyte of interest. Preferably, the urine sample is assayed for NGAL as soon as possible after collection. While pretreatment is not necessary for most urine samples, pretreatment optionally can be done for mere convenience (e.g., as part of a regimen on a commercial platform) or another reason. The pretreatment reagent can be any reagent appropriate for use with the immunoassay and kits of the present disclosure. The pretreatment optionally comprises: (a) one or more solvents (e.g., methanol and ethylene glycol) and salt, (b) one or more solvents, salt and detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., Abbott TDx Monoclonal Antibody Assay Evaluated for Measuring Cyclosporine in Whole Blood, Clin. Chem. 36: 1969-1973 (1990), and Wallemacq et al., Evaluation of the New AxSYM Cyclosporine Assay: Comparison with TDx Monoclonal Whole Blood and EMIT Cyclosporine Assays, Clin. Chem. 45: 432-435 (1999)), and/or as commercially available. Additionally, pretreatment can be done as described in Abbott's U.S. Pat. No. 5,135,875, European Pat. Pub. No. 0 471 293, U.S. Provisional Pat. App. No. 60/878,017 filed Dec. 29, 2006, and now published as U.S. Patent Application Publication No. 2009-0325198, and U.S. Pat. App. Pub. No. 2008/0020401 (all incorporated by reference in their entireties for their teachings regarding pretreatment). The pretreatment reagent can be a heterogeneous agent or a homogeneous agent.

With use of a heterogeneous pretreatment reagent, the pretreatment reagent precipitates analyte binding protein (e.g., protein that can bind to NGAL or a fragment thereof) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of urine sample and pretreatment reagent are contacted with a labeled specific binding partner for NGAL (or a fragment thereof), such as a labeled anti-NGAL monoclonal antibody (or an antigenically reactive fragment thereof). The pretreatment reagent employed for such an assay typically is diluted in the pretreated urine sample mixture, either before or during capture by the first specific binding partner. Despite such dilution, a certain amount of the pretreatment reagent (for example, 5 M methanol and/or 0.6 Methylene glycol) is still present (or remains) in the urine sample mixture during capture.

In a heterogeneous format, after the urine sample is obtained from a subject, a first mixture is prepared. The mixture contains the urine sample being assessed for NGAL (or fragments thereof) and a first specific binding partner, wherein the first specific binding partner and any NGAL contained in the urine sample form a first specific binding partner-NGAL complex. Preferably, the first specific binding partner is an anti-NGAL antibody or a fragment thereof. The order in which the urine sample and the first specific binding partner are added to form the mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip.

After the mixture containing the first specific binding partner-NGAL complex is formed, any unbound (as well as dimeric, trimeric and any oligomeric) NGAL is removed from the complex using any technique known in the art. For example, the unbound NGAL can be removed by washing. Desirably, however, the first specific binding partner is present in excess of any NGAL present in the urine sample, such that all NGAL that is present in the urine sample is bound by the first specific binding partner.

After any unbound NGAL is removed, a second specific binding partner is added to the mixture to form a first specific binding partner-NGAL-second specific binding partner complex. The second specific binding partner is preferably an anti-NGAL antibody that binds to an epitope on NGAL that differs from the epitope on NGAL bound by the first specific binding partner. Moreover, also preferably, the second specific binding partner is labeled with or contains a detectable label as described above.

Any suitable detectable label as is known in the art can be used. For example, the detectable label can be a radioactive label (such as ³H, ¹²⁵I, ³⁵S, ¹⁴C, ³²P, and ³³P), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16: 1324-1328 (2006); Adamczyk et al., Bioorg. Med. Chem. Lett. 4: 2313-2317 (2004); Adamczyk et al., Biorg. Med. Chem. Lett. 14: 3917-3921 (2004); and Adamczyk et al., Org. Lett. 5: 3779-3782 (2003)).

A preferred acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63: 5636-5639 (1998); Adamczyk et al., Tetrahedron 55: 10899-10914 (1999); Adamczyk et al., Org. Lett. 1: 779-781 (1999); Adamczyk et al., Bioconjugate Chem. 11: 714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5: 3779-3782 (2003); and U.S. Pat. Nos. 5,468,646, 5,543,524 and 5,783,699 (each of which is incorporated herein by reference in its entirety for its teachings regarding same).

Another preferred acridinium compound is an acridinium-9-carboxylate aryl ester. An example of an acridinium-9-carboxylate aryl ester of formula II is 10-methyl-9-(phenoxycarbonyl)acridinium fluorosulfonate (available from Cayman Chemical, Ann Arbor, Mich.). Methods for preparing acridinium 9-carboxylate aryl esters are described in McCapra et al., Photochem. Photobiol. 4: 1111-21 (1965); Razavi et al., Luminescence 15: 245-249 (2000); Razavi et al., Luminescence 15: 239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the urine sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

Chemiluminescent assays can be performed in accordance with the methods described in Adamczyk et al., Anal. Chim. Acta 579(1): 61-67 (2006). While any suitable assay format can be used, a microplate chemiluminometer (Mithras LB-940, Berthold Technologies U.S.A., LLC, Oak Ridge, Tenn.) enables the assay of multiple samples of small volumes rapidly. The chemiluminometer can be equipped with multiple reagent injectors using 96-well black polystyrene microplates (Costar #3792). Each sample can be added into a separate well, followed by the simultaneous/sequential addition of other reagents as determined by the type of assay employed. Desirably, the formation of pseudobases in neutral or basic solutions employing an acridinium aryl ester is avoided, such as by acidification. The chemiluminescent response is then recorded well-by-well. In this regard, the time for recording the chemiluminescent response will depend, in part, on the delay between the addition of the reagents and the particular acridinium employed.

The order in which the urine sample and the specific binding partner(s) are added to form the mixture for chemiluminescent assay is not critical. If the first specific binding partner is detectably labeled with an acridinium compound, detectably labeled first specific binding partner-NGAL complexes form. Alternatively, if a second specific binding partner is used and the second specific binding partner is detectably labeled with an acridinium compound, detectably labeled first specific binding partner-NGAL-second specific binding partner complexes form. Any unbound specific binding partner, whether labeled or unlabeled, can be removed from the mixture using any technique known in the art, such as washing.

Hydrogen peroxide can be generated in situ in the mixture or provided or supplied to the mixture before, simultaneously with, or after the addition of an above-described acridinium compound. Hydrogen peroxide can be generated in situ in a number of ways such as would be apparent to one skilled in the art.

Alternatively, a source of hydrogen peroxide can be simply added to the mixture. For example, the source of the hydrogen peroxide can be one or more buffers or other solutions that are known to contain hydrogen peroxide. In this regard, a solution of hydrogen peroxide can simply be added.

Upon the simultaneous or subsequent addition of at least one basic solution to the sample, a detectable signal, namely, a chemiluminescent signal, indicative of the presence of NGAL is generated. The basic solution contains at least one base and has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate, and calcium bicarbonate. The amount of basic solution added to the sample depends on the concentration of the basic solution. Based on the concentration of the basic solution used, one skilled in the art can easily determine the amount of basic solution to add to the sample.

The chemiluminescent signal that is generated can be detected using routine techniques known to those skilled in the art. Based on the intensity of the signal generated, the amount of NGAL in the sample can be quantified. Specifically, the amount of NGAL in the sample is proportional to the intensity of the signal generated. The amount of NGAL present can be quantified by comparing the amount of light generated to a standard curve for NGAL or by comparison to a reference standard. The standard curve can be generated using serial dilutions or solutions of known concentrations of NGAL by mass spectroscopy, gravimetric methods, and other techniques known in the art.

NGAL immunoassays generally can be conducted using any format known in the art, such as, but not limited to, a sandwich format, as further described in U.S. Provisional Patent Application No. 60/981,473 (the '473 application), which was filed on Oct. 19, 2007, has since published as U.S. Patent Application Publication No. 2009-0269777, and which is hereby incorporated by reference. Specifically, in one format at least two antibodies are employed to separate and quantify NGAL, such as human NGAL, or a fragment thereof in a sample. More specifically, the at least two antibodies bind to certain different epitopes on NGAL (or a fragment thereof) forming an immune complex, which is referred to as a “sandwich.” Generally, in the immunoassays one or more antibodies can be used to capture the NGAL (or a fragment thereof) in the urine sample (these antibodies are frequently referred to as a “capture” antibody or “capture” antibodies) and one or more antibodies can be used to bind a detectable (namely, quantifiable) label to the sandwich (these antibodies are frequently referred to as the “detection antibody,” the “detection antibodies,” the “conjugate,” or the “conjugates”).

Generally speaking, a sample being tested for (for example, suspected of containing) NGAL (or a fragment thereof) can be contacted with at least one capture antibody (or antibodies) and at least one detection antibody (which can be a second detection antibody or a third detection antibody) either simultaneously or sequentially and in any order. For example, the urine sample can be first contacted with at least one capture antibody and then (sequentially) with at least one detection antibody. Alternatively, the urine sample can be first contacted with at least one detection antibody and then (sequentially) with at least one capture antibody. In yet another alternative, the urine sample can be contacted simultaneously with a capture antibody and a detection antibody.

In the sandwich assay format, a sample suspected of containing NGAL (or a fragment thereof) is first brought into contact with an at least one first capture antibody under conditions that allow the formation of a first antibody/NGAL complex. If more than one capture antibody is used, a first multiple capture antibody/NGAL complex is formed. In a sandwich assay, the antibodies, preferably, the at least one capture antibody, are used in molar excess amounts of the maximum amount of NGAL (or a fragment thereof) expected in the urine sample. For example, from about 5 μg to about 1 mg of antibody per mL of buffer (e.g., microparticle coating buffer) can be used.

Competitive inhibition immunoassays, which are often used to measure small analytes because binding by only one antibody is required, comprise sequential and classic formats. In a sequential competitive inhibition immunoassay a capture monoclonal antibody to an analyte of interest is coated onto a well of a microtiter plate. When the sample containing the analyte of interest is added to the well, the analyte of interest binds to the capture monoclonal antibody. After washing, a known amount of labeled (e.g., biotin or horseradish peroxidase (HRP)) analyte is added to the well. A substrate for an enzymatic label is necessary to generate a signal. An example of a suitable substrate for HRP is 3,3′,5,5′-tetramethylbenzidine (TMB). After washing, the signal generated by the labeled analyte is measured and is inversely proportional to the amount of analyte in the sample. In a classic competitive inhibition immunoassay a monoclonal antibody to an analyte of interest is coated onto a well of a microtiter plate. However, unlike the sequential competitive inhibition immunoassay, the sample and the labeled analyte are added to the well at the same. Any analyte in the sample competes with labeled analyte for binding to the capture monoclonal antibody. After washing, the signal generated by the labeled analyte is measured and is inversely proportional to the amount of analyte in the sample.

Optionally, prior to contacting the urine sample with the at least one capture antibody (for example, the first capture antibody), the at least one capture antibody can be bound to a solid support, which facilitates the separation of the first antibody/NGAL (or a fragment thereof) complex from the urine sample. The substrate to which the capture antibody is bound can be any suitable solid support or solid phase that facilitates separation of the capture antibody-analyte complex from the sample.

Examples include a well of a plate, such as a microtiter plate, a test tube, a porous gel (e.g., silica gel, agarose, dextran, or gelatin), a polymeric film (e.g., polyacrylamide), beads (e.g., polystyrene beads or magnetic beads), a strip of a filter/membrane (e.g., nitrocellulose or nylon), microparticles (e.g., latex particles, magnetizable microparticles (e.g., microparticles having ferric oxide or chromium oxide cores and homo- or hetero-polymeric coats and radii of about 1-10 microns). The substrate can comprise a suitable porous material with a suitable surface affinity to bind antigens and sufficient porosity to allow access by detection antibodies. A microporous material is generally preferred, although a gelatinous material in a hydrated state can be used. Such porous substrates are preferably in the form of sheets having a thickness of about 0.01 to about 0.5 mm, preferably about 0.1 mm. While the pore size may vary quite a bit, preferably the pore size is from about 0.025 to about 15 microns, more preferably from about 0.15 to about 15 microns. The surface of such substrates can be activated by chemical processes that cause covalent linkage of an antibody to the substrate. Irreversible binding, generally by adsorption through hydrophobic forces, of the antigen or the antibody to the substrate results; alternatively, a chemical coupling agent or other means can be used to bind covalently the antibody to the substrate, provided that such binding does not interfere with the ability of the antibody to bind to NGAL.

Alternatively, the antibody can be bound with microparticles, which have been previously coated with streptavidin or biotin (e.g., using Power-BindTM-SA-MP streptavidin-coated microparticles (Seradyn, Indianapolis, Ind.)) or anti-species-specific monoclonal antibodies. If necessary, the substrate can be derivatized to allow reactivity with various functional groups on the antibody. Such derivatization requires the use of certain coupling agents, examples of which include, but are not limited to, maleic anhydride, N-hydroxysuccinimide, and 1-ethyl-3-(3-dimethylaminopropyl) carbodiimide. If desired, one or more capture reagents, such as antibodies (or fragments thereof), each of which is specific for NGAL can be attached to solid phases in different physical or addressable locations (e.g., such as in a biochip configuration (see, e.g., U.S. Pat. No. 6,225,047, Int'l Pat. App. Pub. No. WO 99/51773; U.S. Pat. No. 6,329,209; Intl Pat. App. Pub. No. WO 00/56934, and U.S. Pat. No. 5,242,828). If the capture reagent is attached to a mass spectrometry probe as the solid support, the amount of NGAL bound to the probe can be detected by laser desorptionionization mass spectrometry. Alternatively, a single column can be packed with different beads, which are derivatized with the one or more capture reagents, thereby capturing the NGAL in a single place (see, antibody derivatized, bead-based technologies, e.g., the xMAP technology of Luminex (Austin, Tex.)).

After the urine sample being assayed for NGAL (or a fragment thereof) is brought into contact with at least one capture antibody (for example, the first capture antibody), the mixture is incubated in order to allow for the formation of a first antibody (or multiple antibody)-NGAL (or a fragment thereof) complex. The incubation can be carried out at a pH of from about 4.5 to about 10.0, at a temperature of from about 2° C. to about 45° C., and for a period from at least about one (1) minute to about eighteen (18) hours, preferably from about 1 to about 24 minutes, most preferably for about 4 to about 18 minutes. The immunoassay described herein can be conducted in one step (meaning the urine sample, at least one capture antibody and at least one detection antibody are all added sequentially or simultaneously to a reaction vessel) or in more than one step, such as two steps, three steps, etc.

After formation of the (first or multiple) capture antibody/NGAL (or a fragment thereof) complex, the complex is then contacted with at least one detection antibody (under conditions which allow for the formation of a (first or multiple) capture antibody/NGAL (or a fragment thereof)/second antibody detection complex). The at least one detection antibody can be the second, third, fourth, etc. antibodies used in the immunoassay. If the capture antibody/NGAL (or a fragment thereof) complex is contacted with more than one detection antibody, then a (first or multiple) capture antibody/NGAL (or a fragment thereof)/(multiple) detection antibody complex is formed. As with the capture antibody (e.g., the first capture antibody), when the at least second (and subsequent) detection antibody is brought into contact with the capture antibody/NGAL (or a fragment thereof) complex, a period of incubation under conditions similar to those described above is required for the formation of the (first or multiple) capture antibody/NGAL (or a fragment thereof)/(second or multiple) detection antibody complex. Preferably, at least one detection antibody contains a detectable label. The detectable label can be bound to the at least one detection antibody (e.g., the second detection antibody) prior to, simultaneously with, or after the formation of the (first or multiple) capture antibody/NGAL (or a fragment thereof)/(second or multiple) detection antibody complex. Any detectable label known in the art can be used (see discussion above, including Polak and Van Noorden (1997) and Haugland (1996)).

The detectable label can be bound to the antibodies either directly or through a coupling agent. An example of a coupling agent that can be used is EDAC (1-ethyl-3-(3-dimethylaminopropyl) carbodiimide, hydrochloride), which is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as CPSP-Acridinium Ester (i.e., 9-[N-tosyl-N-(3-carboxypropyl)]-10-(3-sulfopropyl)acridinium carboxamide) or SPSP-Acridinium Ester (i.e., N10-(3-sulfopropyl)-N-(3-sulfopropyl)-acridinium-9-carboxamide).

The (first or multiple) capture antibody/NGAL/(second or multiple) detection antibody complex can be, but does not have to be, separated from the remainder of the urine sample prior to quantification of the label. For example, if the at least one capture antibody (e.g., the first capture antibody) is bound to a solid support, such as a well or a bead, separation can be accomplished by removing the fluid (of the urine sample) from contact with the solid support. Alternatively, if the at least first capture antibody is bound to a solid support, it can be simultaneously contacted with the NGAL-containing sample and the at least one second detection antibody to form a first (multiple) antibody/NGAL/second (multiple) antibody complex, followed by removal of the fluid (urine sample) from contact with the solid support. If the at least one first capture antibody is not bound to a solid support, then the (first or multiple) capture antibody/NGAL/(second or multiple) detection antibody complex does not have to be removed from the urine sample for quantification of the amount of the label.

After formation of the labeled capture antibody/NGAL/detection antibody complex (e.g., the first capture antibody/NGAL/second detection antibody complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified by detecting the light emitted either visually or by using luminometers, x-ray film, high speed photographic film, a CCD camera, etc. Once the amount of the label in the complex has been quantified, the concentration of NGAL or a fragment thereof in the urine sample is determined by use of a standard curve that has been generated using serial dilutions of NGAL or a fragment thereof of known concentration. Other than using serial dilutions of NGAL or a fragment thereof, the standard curve can be generated gravimetrically, by mass spectroscopy and by other techniques known in the art.

It goes without saying that generation of the standard curve and the NGAL immunoassay generally requires use of some sort of calibrator and/or control. Such calibrators or controls for an NGAL assay are known in the art. An exemplary calibrator/control includes but is not limited to United States Patent Application Nos. US 2009-0123970 and US 2009-0176274 (incorporated by reference for their teachings regarding an NGAL calibrator and/or control).

In a chemiluminescent microparticle assay employing the ARCHITECT® analyzer, the conjugate diluent pH should be about 6.0+/-0.2, the microparticle coating buffer should be maintained at room temperature (i.e., at about 2 to about 8° C.), the microparticle coating buffer pH should be about 5.0+/-0.2, and the microparticle diluent pH should be about 7.0+/-0.2. Solids preferably are less than about 0.2%, such as less than about 0.15%, less than about 0.14%, less than about 0.13%, less than about 0.12%, or less than about 0.11%, such as about 0.10%. When assaying a urine sample for NGAL, the sample volume is preferably low, such as less than or equal to about 10 μL, which is subsequently diluted, whereupon a volume of about 1 μL is tested. In addition, a high ionic strength buffer is preferably used during the first 18 minutes of the two-step assay, which is preferably carried out at a neutral pH, i.e., around pH=7.

FPIAs are based on competitive binding immunoassay principles. A fluorescently labeled compound, when excited by a linearly polarized light, will emit fluorescence having a degree of polarization inversely proportional to its rate of rotation. When a fluorescently labeled tracer-antibody complex is excited by a linearly polarized light, the emitted light remains highly polarized because the fluorophore is constrained from rotating between the time light is absorbed and the time light is emitted. When a “free” tracer compound (i.e., a compound that is not bound to an antibody) is excited by linearly polarized light, its rotation is much faster than the corresponding tracer-antibody conjugate produced in a competitive binding immunoassay. FPIAs are advantageous over RIAs inasmuch as there are no radioactive substances requiring special handling and disposal. In addition, FPIAs are homogeneous assays that can be easily and rapidly performed.

A commercially available anti-NGAL antibody can be used in the methods of assay and kits thereof. Commercially available anti-NGAL antibodies include those available from BioPorto Diagnostics (Gentofte, Denmark), and those available in any NGAL Assay kit for the Abbott ARCHITECT® instrument and marketed by Abbott Laboratories (e.g., Abbott Park, Ill. or Longford, Ireland). Preferably, such commercially available antibodies are used as conjugate/detection antibodies.

Any suitable control composition can be used in the NGAL immunoassays. The control composition generally comprises NGAL and any desirable additives.

In view of the foregoing, the present disclosure provides a method of prognosticating kidney transplant function in a patient is provided. The method comprises comparing the level of uNGAL in the patient at about 24 hours after transplantation with the level of uNGAL in the patient within about 24 hours before transplantation. “Within about 24 hours before transplantation” includes about 24 hours before transplantation (e.g., from 0 to about 24 hours, or from about 3 to about 24 hours, or from about 6 to about 24 hours, before transplantation), about 12 to about 24 hours before transplantation, up to about 12 hours before transplantation (e.g., from 0 to about 12 hours, or from about 3 to about 12 hours, or from about 6 to about 12 hours, before transplantation), up to about 6 hours before transplantation (e.g., from 0 to about 6 hours, or from about 3 to about 6 hours, before transplantation), up to about 5 hours before transplantation (e.g., from 0 to about 5 hours, or from about 3 to about 5 hours, before transplantation), up to about 4 hours before transplantation (e.g., from 0 to about 4 hours, or from about 3 to about 4 hours, before transplantation), up to about 3 hours before transplantation (e.g., from 0 to about 3 hours before transplantation), up to about 2 hours before transplantation (e.g., from 0 to about 2 hours before transplantation), up to about an hour before transplantation (e.g., from 0 to about 1 hour before transplantation), and immediately before transplantation, depending upon a number of variables, such as the availability of a kidney for transplant, the readiness of the patient for transplantation, and the like.

A decrease in the level of uNGAL of less than about 55% (such as about 50%, about 45%, about 40%, about 35%, about 30%, about 25%, about 20%, about 15%, about 10%, or about 5%) predicts delayed graft function (DGF), whereas a decrease in the level of uNGAL of at least about 55% (such as about 60%, about 65%, about 70%, about 75%, about 80%, about 85%, about 90%, or about 95%) predicts early graft function (EGF). A decrease in the level of uNGAL of less than about 55% can be a decrease from greater than 0% to about 55% (e.g., a decrease of from about 5% to about 55%, or from about 10% to about 55%), such as from greater than 0% to about 50% (e.g., a decrease of from about 5% to about 50%, or from about 10% to about 50%), from greater than 0% to about 45% (e.g., a decrease of from about 5% to about 45%, or from about 10% to about 45%), from greater than 0% to about 40% (e.g., a decrease of from about 5% to about 40%, or from about 10% to about 40%), from greater than 0% to about 35% (e.g., a decrease of from about 5% to about 35%, or from about 10% to about 35%), from greater than 0% to about 30% (e.g., a decrease of from about 5% to about 30%, or from about 10% to about 30%), from greater than 0% to about 25% (e.g., a decrease of from about 5% to about 25%, or from about 10% to about 25%), from greater than 0% to about 20% (e.g., a decrease of from about 5% to about 20%, or from about 10% to about 20%), from greater than 0% to about 15% (e.g., a decrease of from about 5% to about 15%, or from about 10% to about 15%), from greater than 0% to about 10% (e.g., a decrease of from about 5% to about 10%), and from greater than 0% to about 5%.

A decrease in the level of uNGAL of at least about 55% can be a decrease from about 55% to about 100% (e.g., a decrease of from about 60% to about 100%, or from about 65% to about 100%, or from about 70% to about 100%), such as from about 55% to about 95% (e.g., a decrease of from about 60% to about 90%, or from about 65% to about 90%, or from about 70% to about 90%), from about 55% to about 90% (e.g., a decrease of from about 60% to about 90%, or from about 65% to about 90%, or from about 70% to about 90%), from about 55% to about 85% (e.g., a decrease of from about 60% to about 85%, or from about 65% to about 85%, or from about 70% to about 85%), from about 55% to about 80% (e.g., a decrease of from about 60% to about 80%, or from about 65% to about 80%, or from about 70% to about 80%), from about 55% to about 75% (e.g., a decrease of from about 60% to about 75%, or from about 65% to about 75%, or from about 70% to about 75%), from about 55% to about 70% (e.g., a decrease of from about 60% to about 70%, or from about 65% to about 70%), from about 55% to about 65% (e.g., a decrease of from about 60% to about 65%), and from about 55% to about 60%. The method can further comprise determining the volume of urine voided by the patient within about 24 hours of transplantation (e.g., from 0 to about 24 hours, or from about 3 to about 24 hours, or from about 6 to about 24 hours, or from about 12 to about 24 hours, of transplantation). A volume of urine that is less than about one liter (such as about 0.90 liter, about 0.85 liter, about 0.80 liter, about 0.75 liter, about 0.70 liter, about 0.65 liter, about 0.60 liter, about 0.55 liter, about 0.50 liter, about 0.45 liter, about 0.40 liter, about 0.35 liter, about 0.30 liter, about 0.25 liter, about 0.20 liter, about 0.15 liter, or about 0.10 liter) indicates DGF, whereas a volume of urine that is greater than about one liter (such as about 1.1 liter, about 1.2 liter, about 1.3 liter, about 1.4 liter, about 1.5 liter, about 1.6 liter, about 1.7 liter, about 1.8 liter, or about 1.9 liter) indicates EGF.

Another method of prognosticating kidney transplant function in a patient is provided. The method comprises comparing the level of uNGAL in the patient at about 24 hours after transplantation with the level of uNGAL in the patient within about 24 hours before transplantation (e.g., from 0 to about 24 hours, or from about 3 to about 24 hours, or from about 6 to about 24 hours, or from about 12 to about 24 hours, before transplantation). A decrease in the level of uNGAL of less than about 20% (such as about 17.5%, about 15%, about 12.5%, about 10%, about 7.5%, or about 5%) predicts DGF lasting more than about 14 days. A decrease in the level of uNGAL of less than about 20% can be a decrease from greater than 0% to about 20% (e.g., a decrease of from about 5% to about 20%, or from about 10% to about 20%), such as from greater than 0% to about 17.5% (e.g., a decrease of from about 5% to about 17.5%, or from about 10% to about 17.5%), from greater than 0% to about 15% (e.g., a decrease of from about 5% to about 15%, or from about 10% to about 15%), from greater than 0% to about 12.5% (e.g., a decrease of from about 5% to about 12.5%, or from about 10% to about 12.5%), from greater than 0% to about 10% (e.g., a decrease of from about 5% to about 10%), from greater than 0% to about 7.5% (e.g., a decrease of from about 5% to about 7.5%), from greater than 0% to about 5%, and from greater than 0% to about 2.5%. The method can further comprise comparing the level of uNGAL in the patient at about 72 hours after transplantation with the level of uNGAL in the patient at about 24 hours after transplantation. An increase in the level of uNGAL from about 24 hours after transplantation to about 72 hours after transplantation further predicts DGF lasting more than about 14 days. “About 72 hours after transplantation” can range, e.g., from 0 to about 72 hours, or from about 12 to about 72 hours, or from about 24 to about 72 hours, or from about 36 to about 72 hours, or from about 48 to about 72 hours, from 0 to about 12 hours, or from about 0 to about 24 hours, or from about 0 to about 36 hours, or from about 0 to about 48 hours, after transplantation. “About 24 hours after transplantation can range”, e.g., from 0 to about 24 hours, or from about 3 to about 24 hours, or from about 6 to about 24 hours, or from about 12 to about 24 hours, or from about 0 to about 3 hours, or from about 0 to about 12 hours, or from about 0 to about 17.5 hours, after transplantation. “More than about 14 days”, can range, e.g., from about 14 to about 28 days

Yet another method of prognosticating kidney transplant function in a patient is provided. The method comprises comparing the level of uNGAL in the patient at about 24 hours after transplantation with the level of uNGAL in the patient within about 24 hours before transplantation. A decrease in the level of uNGAL of at least about 20% and less than about 35% (such as about 21%, about 22%, about 23%, about 24%, about 25%, about 26%, about 27%, about 28%, about 29%, about 30%, about 31%, about 32%, about 33%, or about 34%) predicts DGF lasting less than about 14 days. A decrease in the level of uNGAL of at least about 20% and less than about 35% can be a decrease from about 20% to about 32.5%, from about 20% to about 30%, from about 20% to about 27.5%, from about 20% to about 25%, and from about 20% to about 22.5%. The method can further comprise comparing the level of uNGAL in the patient at about 72 hours after transplantation with the level of uNGAL in the patient at about 24 hours after transplantation. A decrease in the level of uNGAL from about 24 hours after transplantation to about 72 hours after transplantation of at least about 30% (such as about 32.5%, about 35%, about 37.5%, about 40%, about 42.5%, about 45%, about 47.5%, about 50%, about 55%, about 60%, about 65%, about 70%, about 75%, about 80%, about 85%, about 90%, or about 95%) predicts DGF lasting ≦7 days (e.g., lasting either 1, 2, 3, 4, 5, or 6 days), whereas a decrease in the level of uNGAL of less than about 10% (such as about 9%, about 8%, about 7%, about 6%, about 5%, about 4%, about 3%, about 2%, about 1%, from about 0% to about 10%, from about 0% to about 5%, from about 0% to about 7.5%, from about 5% to about 10%, from about 7.5% to about 10%) predicts DGF lasting from about 8 days to about 14 days. A decrease in the level of uNGAL of at least about 30% can be a decrease from about 30% to about 100%, such as a decrease from about 30% to about 95%, a decrease from about 30% to about 90%, a decrease from about 30% to about 85%, a decrease from about 30% to about 80%, a decrease from about 30% to about 75%, a decrease from about 30% to about 70%, a decrease from about 30% to about 65%, a decrease from about 30% to about 60%, a decrease from about 30% to about 55%, a decrease from about 30% to about 50%, a decrease from about 30% to about 45%, a decrease from about 30% to about 40%, and a decrease from about 30% to about 35%. A decrease in the level of uNGAL of less than about 10% further can be a decrease from greater than 0% to about 10%, such as a decrease from greater than 0% to about 7.5%, a decrease from greater than 0% to about 5%, and a decrease from greater than 0% to about 2.5%.

Further provided is a method of prognosticating kidney transplant function in a patient diagnosed with DGF. The method comprises comparing the level of uNGAL in the patient at about 72 hours after transplantation with the level of uNGAL in the patient at about 24 hours after transplantation. A decrease in the level of uNGAL of at least about 30% (such as about 32.5%, about 35%, about 37.5%, about 40%, about 42.5%, about 45%, about 47.5%, about 50%, about 55%, about 60%, about 65%, about 70%, about 75%, about 80%, about 85%, about 90%, or about 95%) predicts DGF lasting ≦7 days (e.g., either about 1, about 2, about 3, about 4, about 5, or about 6 days). A decrease in the level of uNGAL of at least about 30% can be a decrease from about 30% to about 100%, such as a decrease from about 30% to about 95%, a decrease from about 30% to about 90%, a decrease from about 30% to about 85%, a decrease from about 30% to about 80%, a decrease from about 30% to about 75%, a decrease from about 30% to about 70%, a decrease from about 30% to about 65%, a decrease from about 30% to about 60%, a decrease from about 30% to about 55%, a decrease from about 30% to about 50%, a decrease from about 30% to about 45%, a decrease from about 30% to about 40%, and a decrease from about 30% to about 35%.

Still further provided is another method of prognosticating kidney transplant function in a patient diagnosed with DGF. The method comprises comparing the level of uNGAL in the patient at about 72 hours after transplantation with the level of uNGAL in the patient at about 24 hours after transplantation. A decrease in the level of uNGAL of less than about 10% (such as about 9%, about 8%, about 7%, about 6%, about 5%, about 4%, about 3%, about 2%, or about 1%) predicts DGF lasting from about 8 days to about 14 days. A decrease in the level of uNGAL of less than about 10% can be a decrease from greater than 0% to about 10%, such as a decrease from greater than 0% to about 7.5%, a decrease from greater than 0% to about 5%, and a decrease from greater than 0% to about 2.5%. As previously described, “72 hours after transplantation” can range, e.g., from 0 to about 72 hours, or from about 12 to about 72 hours, or from about 24 to about 72 hours, or from about 36 to about 72 hours, or from about 48 to about 72 hours, from 0 to about 12 hours, or from about 0 to about 24 hours, or from about 0 to about 36 hours, or from about 0 to about 48 hours, after transplantation. Likewise, “about 24 hours after transplantation” can range, e.g., from 0 to about 24 hours, or from about 3 to about 24 hours, or from about 6 to about 24 hours, or from about 12 to about 24 hours, or from about 0 to about 3 hours, or from about 0 to about 12 hours, or from about 0 to about 17.5 hours, after transplantation.

Even still further provided is another method of prognosticating kidney transplant function in a patient diagnosed with DGF. The method comprises comparing the level of uNGAL in the patient at about 72 hours after transplantation (as previously described) with the level of uNGAL in the patient at about 24 hours after transplantation (as previously described). An increase in the level of uNGAL from about 24 hours after transplantation to about 72 hours after transplantation predicts DGF lasting longer than about 14 days.

A method of assessing risk of DGF in a patient, who has been diagnosed with EGF based on (i) the volume of urine voided by the patient within about 24 hours of transplantation being at least about one liter and/or (ii) the level of creatinine in the plasma of the patient at about 24 hours after transplantation being lower than the level of creatinine in the plasma of the patient within about 24 hours before transplantation, is also provided. The method comprises comparing the level of uNGAL in the patient at about 24 hours after transplantation with the level of uNGAL in the patient within about 24 hours before transplantation. A decrease in the level of uNGAL of less than about 55% (such as about 50%, about 45%, about 40%, about 35%, about 30%, about 25%, about 20%, about 15%, about 10%, or about 5%) predicts risk of DGF. A decrease in the level of uNGAL of less than about 55% can be a decrease from greater than 0% to about 55%, such as from greater than 0% to about 50%, from greater than 0% to about 45%, from greater than 0% to about 40%, from greater than 0% to about 35%, from greater than 0% to about 30%, from greater than 0% to about 25%, from greater than 0% to about 20%, from greater than 0% to about 15%, from greater than 0% to about 10%, and from greater than 0% to about 5%.

### Anti-NGAL Antibody

Any anti-NGAL antibody appropriate for use in the methods as described herein can be employed in the context of the present disclosure. Examplary antibodies include but are not limited to: those present in an NGAL Assay kit for the Abbott ARCHITECT® instrument (Abbott Laboratories, Abbott Ireland, Longford, Ireland, and soon to be marketed, by Abbott Laboratories, Abbott Park, Ill.); an antibody produced by murine hybridoma cell line 1-2322-455 having ATCC Accession No. PTA-8024 and an antibody produced by murine hybridoma cell line 1-903-430 having ATCC Accession No. PTA-8026; those described in United States Patent Application Nos. US 2009-0263894 and US 2009-0124022 (incorporated by reference for their teachings regarding same).

### Antibody Production

Antibodies (or fragments thereof) that specifically bind to NGAL (or a fragment thereof) can be made using a variety of different techniques known in the art. For example, polyclonal and monoclonal antibodies can be raised by immunizing a suitable subject (such as, but not limited to, a rabbit, a goat, a mouse, or other mammal) with an immunogenic preparation, which contains a suitable immunogen. The immunogen can be enriched/purified and isolated from a cell that produces it using affinity chromatography, immune-precipitation or other techniques, which are well-known in the art. Alternatively, immunogen can be prepared using chemical synthesis using routine techniques known in the art (such as, but not limited to, a synthesizer). The antibodies raised in the subject can then be screened to determine if the antibodies bind to the immunogen (or a fragment thereof).

The unit dose of immunogen (namely, the purified protein, tumor cell expressing the protein, or recombinantly expressed immunogen (or a fragment or a variant (or a fragment thereof) thereof) and the immunization regimen will depend upon the subject to be immunized, its immune status, and the body weight of the subject. To enhance an immune response in the subject, an immunogen can be administered with an adjuvant, such as Freund's complete or incomplete adjuvant.

Immunization of a subject with an immunogen as described above induces a polyclonal antibody response. The antibody titer in the immunized subject can be monitored over time by standard techniques such as an ELISA using an immobilized antigen.

Other methods of raising antibodies include using transgenic mice, which express human immunoglobin genes (see, for example, Intl Pat. App. Pub. Nos. WO 91/00906, WO 91/10741, and WO 92/03918). Alternatively, human monoclonal antibodies can be produced by introducing an antigen into immune-deficient mice that have been engrafted with human antibody-producing cells or tissues (for example, human bone marrow cells, peripheral blood lymphocytes (PBL), human fetal lymph node tissue, or hematopoietic stem cells). Such methods include raising antibodies in SCID-hu mice (see, for example, Int'l Pat. App. Pub. No. WO 93/05796; U.S. Pat. No. 5,411,749; or McCune et al., Science 241: 1632-1639 (1988)) or Rag-1/Rag-2 deficient mice. Human antibody-immune deficient mice are also commercially available. For example, Rag-2 deficient mice are available from Taconic Farms (Germantown, N.Y.).

Monoclonal antibodies can be generated by immunizing a subject with an immunogen. At the appropriate time after immunization, for example, when the antibody titers are at a sufficiently high level, antibody-producing cells can be harvested from an immunized animal and used to prepare monoclonal antibodies using standard techniques. For example, the antibody-producing cells can be fused by standard somatic cell fusion procedures with immortalizing cells, such as myeloma cells, to yield hybridoma cells. Such techniques are well-known in the art, and include, for example, the hybridoma technique as originally developed by Kohler and Milstein, Nature 256: 495-497 (1975)), the human B cell hybridoma technique (Kozbar et al., Immunology Today 4: 72 (1983)), and the Epstein-Ban virus (EBV)-hybridoma technique to produce human monoclonal antibodies (Cole et al., Monoclonal Antibodies and Cancer Therapy, Alan R. Liss, Inc. pp. 77-96 (1985)). The technology for producing monoclonal antibody hybridomas is well-known to those skilled in the art.

Monoclonal antibodies also can be made by harvesting antibody-producing cells, for example, splenocytes, from transgenic mice, which express human immunoglobulin genes and which have been immunized with the immunogen. The splenocytes can be immortalized through fusion with human myelomas or through transformation with EBV. These hybridomas can be made using human B cell- or EBV-hybridoma techniques described in the art (See, for example, Boyle et al., European Pat. Pub. No. 0 614 984).

Hybridoma cells producing a monoclonal antibody, which specifically binds to the immunogen, are detected by screening the hybridoma culture supernatants by, for example, screening to select antibodies that specifically bind to the immobilized immunogen (or a fragment thereof), or by testing the antibodies as described herein to determine if the antibodies have the desired characteristics, namely, the ability to bind to immunogen (or a fragment thereof). After hybridoma cells are identified that produce antibodies of the desired specificity, the clones may be subcloned, e.g., by limiting dilution procedures, for example the procedure described by Wands et al. (Gastroenterology 80: 225-232 (1981)), and grown by standard methods.

Hybridoma cells that produce monoclonal antibodies that test positive in the screening assays described herein can be cultured in a nutrient medium under conditions and for a time sufficient to allow the hybridoma cells to secrete the monoclonal antibodies into the culture medium, to thereby produce whole antibodies. Tissue culture techniques and culture media suitable for hybridoma cells are generally described in the art (See, for example, R. H. Kenneth, in Monoclonal Antibodies: A New Dimension In Biological Analyses, Plenum Publishing Corp., New York, N.Y. (1980)). Conditioned hybridoma culture supernatant containing the antibody can then be collected. The monoclonal antibodies secreted by the subclones optionally can be isolated from the culture medium by conventional immunoglobulin purification procedures such as, for example, protein A chromatography, hydroxylapatite chromatography, gel electrophoresis, dialysis, or affinity chromatography.

Monoclonal antibodies can be engineered by constructing a recombinant combinatorial immunoglobulin library and screening the library with the immunogen or a fragment thereof. Kits for generating and screening phage display libraries are commercially available (See, for example, the Pharmacia Recombinant Phage Antibody System, Catalog No. 27-9400-01; and the Stratagene SurfZAP Phage Display Kit, Catalog No. 240612). Likewise, yeast display vectors are known in the art and are commercially available (for example, pYD1 available from Invitrogen). Briefly, the antibody library is screened to identify and isolate phages or yeast cells that express an antibody that specifically binds to the immunogen or a fragment thereof. Preferably, the primary screening of the library involves screening with an immobilized immunogen or a fragment thereof.

Following screening, the display phage or yeast is isolated and the polynucleotide encoding the selected antibody can be recovered from the display phage or yeast (for example, from the phage or yeast genome) and subcloned into other expression vectors (e.g., into Saccharomyces cerevesiae cells, for example EBY100 cells (Invitrogen)) by well-known recombinant DNA techniques. The polynucleotide can be further manipulated (for example, linked to nucleic acid encoding additional immunoglobulin domains, such as additional constant regions) and/or expressed in a host cell.

Once a monoclonal antibody that specifically binds to NGAL is obtained in accordance with methods described above, it can be sequenced in accordance with methods known in the art. The antibody then can be made using recombinant DNA technology, chemical synthesis, or a combination of chemical synthesis and recombinant DNA technology as described herein.

Furthermore, in some aspects of the disclosure, it may be possible to employ commercially available anti-NGAL antibodies, e.g., as conjugate/detection antibodies, or methods for production of anti-NGAL antibodies as described in the literature. These include, but are not limited to, those available from BioPorto Diagnostics (Gentofte, Denmark).

### Synthetic Production

Once sequenced, polypeptides, such as a monoclonal antibody (or a fragment thereof), which specifically binds to NGAL, can be synthesized using methods known in the art, such as, for example, exclusive solid phase synthesis, partial solid phase synthesis, fragment condensation, and classical solution synthesis. See, e.g., Merrifield, J. Am. Chem. Soc. 85: 2149 (1963). On solid phase, the synthesis typically begins from the C-terminal end of the peptide using an alpha-amino protected resin. A suitable starting material can be prepared, for instance, by attaching the required alpha-amino acid to a chloromethylated resin, a hydroxymethyl resin, or a benzhydrylamine resin. One such chloromethylated resin is sold under the tradename BIO-BEADS SX-1 by Bio Rad Laboratories (Richmond, Calif.), and the preparation of the hydroxymethyl resin is described by Bodonszky et al., Chem. Ind. (London) 38: 1597 (1966). The benzhydrylamine (BHA) resin has been described by Pietta and Marshall, Chem. Comm. 650 (1970) and is commercially available from Beckman Instruments, Inc. (Palo Alto, Calif.) in the hydrochloride form. Automated peptide synthesizers are commercially available, as are services that make peptides to order.

Thus, the polypeptides can be prepared by coupling an alpha-amino protected amino acid to the chloromethylated resin with the aid of, for example, cesium bicarbonate catalyst, according to the method described by Gisin, Helv. Chim. Acta. 56: 1467 (1973). After the initial coupling, the alpha-amino protecting group is removed by a choice of reagents including trifluoroacetic acid (TFA) or hydrochloric acid (HCl) solutions in organic solvents at room temperature.

Suitable alpha-amino protecting groups include those known to be useful in the art of stepwise synthesis of peptides. Examples of alpha-amino protecting groups are: acyl type protecting groups (e.g., formyl, trifluoroacetyl, and acetyl), aromatic urethane type protecting groups (e.g., benzyloxycarbonyl (Cbz) and substituted Cbz), aliphatic urethane protecting groups (e.g., t-butyloxycarbonyl (Boc), isopropyloxycarbonyl, and cyclohexyloxycarbonyl), and alkyl type protecting groups (e.g., benzyl and triphenylmethyl). Boc and Fmoc are preferred protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (DCC) in solution, for example, in methylene chloride and dimethyl formamide (DMF) mixtures.

After the desired amino acid sequence has been completed, the desired peptide is decoupled from the resin support by treatment with a reagent, such as TFA or hydrogen fluoride (HF), which not only cleaves the peptide from the resin, but also cleaves all remaining side chain protecting groups. When the chloromethylated resin is used, HF treatment results in the formation of the free peptide acids. When the benzhydrylamine resin is used, HF treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride to give the free amides, alkylamides, or dialkylamides.

These and other solid phase peptide synthesis procedures are well-known in the art. Such procedures are also described by Stewart and Young in Solid Phase Peptide Syntheses (2nd Ed., Pierce Chemical Company, 1984).

### Recombinant Production

A polypeptide, such as a monoclonal antibody (or a fragment thereof), which specifically binds to NGAL, can be recombinantly produced using methods known in the art. For example, an isolated nucleic acid comprising a nucleotide sequence encoding the antibody (or a fragment thereof) can be expressed in a host cell, and the antibody can be isolated. The isolated nucleic acid can comprise a nucleotide sequence encoding the amino acid sequence of a VH domain region and/or a nucleotide sequence encoding the amino acid sequence of a VL domain region.

A polypeptide, such as a monoclonal antibody (or a fragment thereof), which binds to NGAL, also can be recombinantly produced using methods known in the art. The isolated nucleic acid can comprise a nucleotide sequence encoding the amino acid sequence of a VH domain region and/or a nucleotide sequence encoding the amino acid sequence of a VL domain region.

The isolated nucleic acid can be synthesized with an oligonucleotide synthesizer, for example. One of ordinary skill in the art will readily appreciate that, due to the degeneracy of the genetic code, more than one nucleotide sequence can encode a given amino acid sequence. In this regard, a nucleotide sequence encoding an amino acid sequence that is substantially identical to an amino acid sequence of a VH domain region and/or an amino acid sequence that is substantially identical to an amino acid sequence of a VL domain region can be used, provided that the variant antibody as expressed competes with the antibody comprising the amino acid sequence of a VH domain region and/or the amino acid sequence of a VL domain region for the same epitope on NGAL. Codons, which are favored by a given host cell, preferably are selected for recombinant production. A nucleotide sequence encoding the amino acid sequence of a VH domain region and/or a nucleotide sequence encoding the amino acid sequence of a VL domain region can be combined with other nucleotide sequences using polymerase chain reaction (PCR), ligation, or ligation chain reaction (LCR) to encode an anti-NGAL antibody or antigenically reactive fragment thereof. The individual oligonucleotides typically contain 5′ or 3′ overhangs for complementary assembly. Once assembled, the nucleotide sequence encoding an anti-NGAL antibody or antigenically reactive fragment thereof can be inserted into a vector, operably linked to control sequences as necessary for expression in a given host cell, and introduced (such as by transformation or transfection) into a host cell. The nucleotide sequence can be further manipulated (for example, linked to one or more nucleotide sequences encoding additional immunoglobulin domains, such as additional constant regions) and/or expressed in a host cell.

Although not all vectors and expression control sequences may function equally well to express a polynucleotide sequence of interest and not all hosts function equally well with the same expression system, it is believed that those skilled in the art will be able to make a selection among these vectors, expression control sequences, optimized codons, and hosts for use in the present disclosure without any undue experimentation. For example, in selecting a vector, the host must be considered because the vector must be able to replicate in it or be able to integrate into the chromosome. The vector's copy number, the ability to control that copy number, and the expression of any other proteins encoded by the vector, such as antibiotic markers, should also be considered. In selecting an expression control sequence, a variety of factors also can be considered. These include, but are not limited to, the relative strength of the sequence, its controllability, and its compatibility with the nucleotide sequence encoding the anti-NGAL antibody, particularly with regard to potential secondary structures. Hosts should be selected by consideration of their compatibility with the chosen vector, their codon usage, their secretion characteristics, their ability to fold the polypeptide correctly, their fermentation or culture requirements, their ability (or lack thereof) to glycosylate the protein, and the ease of purification of the products encoded by the nucleotide sequence, etc.

The recombinant vector can be an autonomously replicating vector, namely, a vector existing as an extrachromosomal entity, the replication of which is independent of chromosomal replication (such as a plasmid). Alternatively, the vector can be one which, when introduced into a host cell, is integrated into the host cell genome and replicated together with the chromosome(s) into which it has been integrated.

The vector is preferably an expression vector in which the polynucleotide sequence encoding the anti-NGAL antibody is operably linked to additional segments required for transcription of the polynucleotide sequence. The vector is typically derived from plasmid or viral DNA. A number of suitable expression vectors for expression in the host cells mentioned herein are commercially available or described in the literature. Useful expression vectors for eukaryotic hosts, include, but are not limited to, vectors comprising expression control sequences from SV40, bovine papilloma virus, adenovirus and cytomegalovirus. Specific vectors include pcDNA3.1 (+)\Hyg (Invitrogen Corp., Carlsbad, Calif.) and pCI-neo (Stratagene, La Jolla, Calif.). Examples of expression vectors for use in yeast cells include, but are not limited to, the 2μ plasmid and derivatives thereof, the POT1 vector (see, e.g., U.S. Pat. No. 4,931,373), the pJSO37 vector (described in Okkels, Ann. New York Acad. Sci. 782: 202-207 (1996)) and pPICZ A, B or C (Invitrogen). Examples of expression vectors for use in insect cells include, but are not limited to, pVL941, pBG311 (Cate et al., Cell 45: 685-698 (1986)), and pBluebac 4.5 and pMelbac (both of which are available from Invitrogen).

Other vectors that can be used allow the nucleotide sequence encoding the anti-NGAL antibody to be amplified in copy number. Such amplifiable vectors are well-known in the art. These vectors include, but are not limited to, those vectors that can be amplified by dihydrofolate reductase (DHFR) amplification (see, for example, Kaufman, U.S. Pat. No. 4,470,461; and Kaufman et al., Mol. Cell. Biol. 2: 1304-1319 (1982)) and glutamine synthetase (GS) amplification (see, for example, U.S. Pat. No. 5,122,464 and European Pat. App. Pub. No. 0 338 841).

The recombinant vector can further comprise a nucleotide sequence enabling the vector to replicate in the host cell in question. An example of such a sequence for use in a mammalian host cell is the SV40 origin of replication. Suitable sequences enabling the vector to replicate in a yeast cell are the yeast plasmid 2μ replication genes REP 1-3 and origin of replication.

The vector can also comprise a selectable marker, namely, a gene or polynucleotide, the product of which complements a defect in the host cell, such as the gene coding for DHFR or the Schizosaccharomyces pombe TPI gene (see, e.g., Russell, Gene 40: 125-130 (1985)), or one which confers resistance to a drug, such as ampicillin, kanamycin, tetracycline, chloramphenicol, neomycin, hygromycin or methotrexate. For filamentous fungi, selectable markers include, but are not limited to, amdS, pyrG, arcB, niaD and sC.

Also present in the vector are “control sequences,” which are any components that are necessary or advantageous for the expression of the anti-NGAL antibody. Each control sequence can be native or foreign to the nucleotide sequence encoding the anti-NGAL antibody. Such control sequences include, but are not limited to, a leader, a polyadenylation sequence, a propeptide sequence, a promoter, an enhancer or an upstream activating sequence, a signal peptide sequence, and a transcription terminator. At a minimum, the control sequences include at least one promoter operably linked to the polynucleotide sequence encoding the anti-NGAL antibody.

By “operably linked” is meant the covalent joining of two or more nucleotide sequences, by means of enzymatic ligation or otherwise, in a configuration relative to one another such that the normal function of the sequences can be performed. For example, a nucleotide sequence encoding a presequence or secretory leader is operably linked to a nucleotide sequence for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in the same reading frame. Linking is accomplished by ligation at convenient restriction sites. If such sites do not exist, then synthetic oligonucleotide adaptors or linkers can be used, in conjunction with standard recombinant DNA methods.

A wide variety of expression control sequences can be used in the context of the present disclosure. Such useful expression control sequences include the expression control sequences associated with structural genes of the foregoing expression vectors as well as any sequence known to control the expression of genes of prokaryotic or eukaryotic cells or their viruses, and various combinations thereof. Examples of suitable control sequences for directing transcription in mammalian cells include the early and late promoters of SV40 and adenovirus, for example, the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, J. Mol. Biol. 196: 947-50 (1987)).

In order to improve expression in mammalian cells a synthetic intron can be inserted in the 5′ untranslated region of a polynucleotide sequence encoding the antibody or a fragment thereof. An example of a synthetic intron is the synthetic intron from the plasmid pCI-Neo (available from Promega Corporation, Madison, Wis.).

Examples of suitable control sequences for directing transcription in insect cells include, but are not limited to, the polyhedrin promoter, the P10 promoter, the baculovirus immediate early gene 1 promoter, the baculovirus 39K delayed-early gene promoter, and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4-c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulas glucoamylase, A. nidulans acetamidase, Rhizomucor miehei aspartic proteinase or lipase, the TPI1 terminator, and the ADH3 terminator.

The polynucleotide sequence encoding the antibody of interest may or may not also include a polynucleotide sequence that encodes a signal peptide. The signal peptide is present when the anti-NGAL antibody is to be secreted from the cells in which it is expressed. Such signal peptide, if present, should be one recognized by the cell chosen for expression of the polypeptide. The signal peptide can be homologous or heterologous to the anti-NGAL monoclonal antibody or can be homologous or heterologous to the host cell, i.e., a signal peptide normally expressed from the host cell or one which is not normally expressed from the host cell. Accordingly, the signal peptide can be prokaryotic, for example, derived from a bacterium, or eukaryotic, for example, derived from a mammalian, insect, filamentous fungal, or yeast cell.

The presence or absence of a signal peptide will, for example, depend on the expression host cell used for the production of the anti-NGAL antibody. For use in filamentous fungi, the signal peptide can conveniently be derived from a gene encoding an Aspergillus sp. amylase or glucoamylase, a gene encoding a Rhizomucor miehei lipase or protease or a Humicola lanuginosa lipase. For use in insect cells, the signal peptide can be derived from an insect gene (see, e.g., Int'l Pat. App. Pub. No. WO 90/05783), such as the lepidopteran Manduca sexta adipokinetic hormone precursor (see, e.g., U.S. Pat. No. 5,023,328), the honeybee melittin (Invitrogen), ecdysteroid UDP glucosyltransferase (egt) (Murphy et al., Protein Expression and Purification 4: 349-357 (1993), or human pancreatic lipase (hpl) (Methods in Enzymology 284: 262-272 (1997)).

Specific examples of signal peptides for use in mammalian cells include murine Ig kappa light chain signal peptide (Coloma, J. 1 mm. Methods 152: 89-104 (1992)). Suitable signal peptides for use in yeast cells include the α-factor signal peptide from S. cerevisiae (see, e.g., U.S. Pat. No. 4,870,008), the signal peptide of mouse salivary amylase (see, e.g., Hagenbuchle et al., Nature 289: 643-646 (1981)), a modified carboxypeptidase signal peptide (see, e.g., Valls et al., Cell 48: 887-897 (1987)), the yeast BAR1 signal peptide (see, e.g., Int'l Pat. App. Pub. No. WO 87/02670), and the yeast aspartic protease 3 (YAP3) signal peptide (see, e.g., Egel-Mitani et al., Yeast 6: 127-137 (1990)).

Any suitable host can be used to produce the anti-NGAL antibody, including bacteria, fungi (including yeasts), plant, insect, mammal or other appropriate animal cells or cell lines, as well as transgenic animals or plants. Examples of bacterial host cells include, but are not limited to, gram-positive bacteria, such as strains of Bacillus, for example, B. brevis or B. subtilis, Pseudomonas or Streptomyces, or gram-negative bacteria, such as strains of E. coli. The introduction of a vector into a bacterial host cell can, for instance, be effected by protoplast transformation (see, for example, Chang et al., Molec. Gen. Genet. 168: 111-115 (1979)), using competent cells (see, for example, Young et al., J. of Bacteriology 81: 823-829 (1961), or Dubnau et al., J. of Molec. Biol. 56: 209-221 (1971)), electroporation (see, for example, Shigekawa et al., Biotechniques 6: 742-751 (1988)), or conjugation (see, for example, Koehler et al., J. of Bacteriology 169: 5771-5278 (1987)).

Examples of suitable filamentous fungal host cells include, but are not limited to, strains of Aspergillus, for example, A. oryzae, A. niger, or A. nidulans, Fusarium or Trichoderma. Fungal cells can be transformed by a process involving protoplast formation, transformation of the protoplasts, and regeneration of the cell wall using techniques known to those ordinarily skilled in the art. Suitable procedures for transformation of Aspergillus host cells are described in European Pat. App. Pub. No. 238 023 and U.S. Pat. No. 5,679,543. Suitable methods for transforming Fusarium species are described by Malardier et al., Gene 78: 147-156 (1989), and Int'l Pat. App. Pub. No. WO 96/00787. Yeast can be transformed using the procedures described by Becker and Guarente, In Abelson, J. N. and Simon, M. I., editors, Guide to Yeast Genetics and Molecular Biology, Methods in Enzymology 194: 182-187, Academic Press, Inc., New York; Ito et al, J. of Bacteriology 153: 163 (1983); and Hinnen et al., PNAS USA 75: 1920 (1978).

Examples of suitable yeast host cells include strains of Saccharomyces, for example, S. cerevisiae, Schizosaccharomyces, Klyveromyces, Pichia, such as P. pastoris or P. methanolica, Hansenula, such as H. polymorpha or yarrowia. Methods for transforming yeast cells with heterologous polynucleotides and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters 99: 193-198 (1992), Manivasakam et al., Nucleic Acids Research 21: 4414-4415 (1993), and Ganeva et al., FEMS Microbiology Letters 121: 159-164 (1994).

Examples of suitable insect host cells include, but are not limited to, a Lepidoptora cell line, such as Spodoptera frugiperda (Sf9 or Sf21) or Trichoplusia ni cells (High Five) (see, e.g., U.S. Pat. No. 5,077,214). Transformation of insect cells and production of heterologous polypeptides are well-known to those skilled in the art.

Examples of suitable mammalian host cells include Chinese hamster ovary (CHO) cell lines, simian (e.g., Green Monkey) cell lines (COS), mouse cells (for example, NS/O), baby hamster kidney (BHK) cell lines, human cells (such as human embryonic kidney (HEK) cells (e.g., HEK 293 cells (A.T.C.C. Accession No. CRL-1573))), myeloma cells that do not otherwise produce immunoglobulin protein, and plant cells in tissue culture. Preferably, the mammalian host cells are CHO cell lines and HEK 293 cell lines. Another preferred host cell is the B3.2 cell line (e.g., Abbott Laboratories, Abbott Bioresearch Center), or another dihydrofolate reductase deficient (DHFR⁻) CHO cell line (e.g., available from Invitrogen).

Methods for introducing exogenous polynucleotides into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamine™ 2000. These methods are well-known in the art and are described, for example, by Ausbel et al. (eds.), Current Protocols in Molecular Biology, John Wiley & Sons, New York, USA (1996). The cultivation of mammalian cells is conducted according to established methods, e.g., as disclosed in Jenkins, Ed., Animal Cell Biotechnology, Methods and Protocols, Human Press Inc. Totowa, N.J., USA (1999), and Harrison and Rae, General Techniques of Cell Culture, Cambridge University Press (1997).

In the production methods, cells are cultivated in a nutrient medium suitable for production of the anti-NGAL antibody using methods known in the art. For example, cells are cultivated by shake flask cultivation, small-scale or large-scale fermentation (including continuous, batch, fed-batch, or solid state fermentations) in laboratory or industrial fermenters performed in a suitable medium and under conditions allowing the anti-human NGAL monoclonal antibody to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection). If the anti-NGAL antibody is secreted into the nutrient medium, it can be recovered directly from the medium. If the anti-NGAL antibody is not secreted, it can be recovered from cell lysates.

The resulting anti-NGAL antibody can be recovered by methods known in the art. For example, the anti-NGAL antibody can be recovered from the nutrient medium by conventional procedures including, but not limited to, centrifugation, filtration, extraction, spray drying, evaporation, or precipitation.

The anti-NGAL antibody can be purified by a variety of procedures known in the art including, but not limited to, chromatography (such as, but not limited to, ion exchange, affinity, hydrophobic, chromatofocusing, and size exclusion), electrophoretic procedures (such as, but not limited to, preparative isoelectric focusing), differential solubility (such as, but not limited to, ammonium sulfate precipitation), SDS-PAGE, or extraction (see, for example, Janson and Ryden, editors, Protein Purification, VCH Publishers, New York (1989)).

Antibody fragments are also contemplated. For example, the antibody fragment can include, but is not limited to, a Fab, a Fab′, a Fab′-SH fragment, a di-sulfide linked Fv, a single chain Fv (scFv) and a F(ab′)₂ fragment. Various techniques are known to those skilled in the art for the production of antibody fragments. For example, such fragments can be derived via proteolytic digestion of intact antibodies (see, for example, Morimoto et al., J. Biochem. Biophys. Methods 24: 107-117 (1992), and Brennan et al., Science 229: 81 (1985)) or produced directly by recombinant host cells. For example, Fab′-SH fragments can be directly recovered from E. coli and chemically coupled to form F(ab′)₂ fragments (see, e.g., Carter et al., Bio/Technology 10: 163-167 (1992)). In another embodiment, the F(ab′)₂ is formed using the leucine zipper GCN4 to promote assembly of the F(ab′)₂ molecule. Alternatively, Fv, Fab or F(ab′)₂ fragments can be isolated directly from recombinant host cell culture. Single chain variable region fragments (scFv) are made by linking light and/or heavy chain variable regions by using a short linking peptide or sequence (see, e.g., Bird et al., Science 242: 423-426 (1998)). The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art. Moreover, other forms of single-chain antibodies, such as diabodies are also contemplated by the present disclosure. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see, for example, Holliger et al., PNAS USA 90: 6444-6448 (1993); and Poljak et al., Structure 2: 1121-1123 (1994)).

The antibody and antigenically reactive fragment thereof have a variety of uses. In one aspect, the antibody (or a fragment thereof) can be used as one or more immunodiagnostic reagents. For example, the antibodies of the present disclosure can be used as one or more immunodiagnostic reagents in one or more methods for detecting the level of NGAL in a urine sample. More specifically, the antibody (or antigenically reactive fragment thereof) can be used as a capture antibody or a detection antibody in an immunoassay to detect the presence of NGAL, such as human NGAL (or a fragment thereof), in a urine sample.

### Kit

In view of the above, a kit is provided. The kit comprises at least one component for assaying urine from a patient, who has received a kidney transplant or is about to receive a kidney transplant, for NGAL. The kit further comprises instructions for assaying the urine for NGAL and assessing kidney transplant function in the patient.

Also in view of the above, another kit is provided. The kit comprises at least one component for assaying urine from a patient, who has received a kidney transplant and has been diagnosed with EGF, for NGAL. The kit further comprises instructions for assaying the urine for NGAL and assessing risk of DGF in the patient.

The kit can comprise instructions, for example, for assaying the urine sample for NGAL (or a fragment thereof) by immunoassay, e.g., chemiluminescent microparticle immunoassay. The instructions can be in paper form or computer-readable form, such as a disk, CD, DVD, or the like. The antibody can be a NGAL capture antibody and/or a NGAL detection antibody. Alternatively or additionally, the kit can comprise a calibrator or control, e.g., purified, and optionally lyophilized, NGAL (or a fragment thereof), and/or at least one container (e.g., tube, microtiter plates or strips, which can be already coated with an anti-NGAL monoclonal antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve or a reference standard for purposes of quantifying NGAL.

Any antibodies, which are provided in the kit, such as recombinant antibodies specific for NGAL, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes or reagents for detecting the analyte. The antibodies, calibrators and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a urine sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine sample). Where appropriate, the kit optionally also can contain reaction vessels, mixing vessels, and other components that facilitate the preparation of reagents or the urine sample. The kit can also include one or more instrument for assisting with obtaining a urine sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc or chip.

### Adaptation of Kit and Method

The kit (or components thereof), as well as the method of determining the concentration of NGAL in a urine sample by an assay as described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours), an automated or semi-automated format (e.g., ARCHITECT®, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing NGAL is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, NGAL, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of analyte (NGAL) in the sample by means of an embedded algorithm and factory-determined calibration curve.

It further goes without saying that the methods and kits as described herein necessarily encompass other reagents and methods for carrying out the assay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent.

The following example serves to illustrate the present disclosure. The example is not intended to limit the scope of the claimed invention in any way.

### Example

Consecutive adult, dialysis-dependent, recipients (n=176; all Caucasian except for one) of kidneys transplanted from deceased donors were recruited for the clinical study described herein. None of the donors had diabetes, and all donors were Caucasian and received intravenous steroids before the organ retrieval operation. Mannitol was given to the donors before the in situ perfusion. University of Wisconsin solution (“UW,” a well-known organ and biological tissue cold storage solution used for preservation) was used for in situ perfusion and cold-storage preservation of the kidneys. Calcineurin inhibitor (cyclosporine, n=135; tacrolimus, n=41) was given orally to recipients before tranplantation, and continued after transplantation with a target level of 200-250 μg/mL for cyclosporine and a target level of 6-12 μg/mL for tacrolimus. Other immunosuppression consisted of mycophenolate mofetil and steroids. Donor characteristics are shown in Table 1. Recipient characteristics are shown in Table 2.

TABLE 1

| | |
|---|---|
| Number (N) | 99 |
| Mean age, years (range) | 51.8 (9-75) |
| Gender | |
| Female | 43 (43.4%) |
| Male | 56 (56.6%) |
| Cause of death | |
| Cerebrovascular accident | 74 (74.7%) |
| Traumatic brain injury | 25 (25.3%) |
| Mean plasma creatinine, μmol/L (range) | 62 (28-143) |
| Mean eGFR, mL/min (range) | 122 (60-263) |
| History of hypertension | 27 (27.3%) |
| Need for cardiopulmonary resuscitation | 21 (21.2%) |
| Ante mortem intracranial surgery | 30 (30.3%) |
| Use of inotropes | 87 (87.9%) |
| Use of anti-diuretic hormone | 60 (60.6%) |
| Multi-organ donor | 56 (56.6%) |
| Mean hospital days before brain death | 1.9 (1-14) |
| (range) | |

eGFR = estimated glomerular filtration rate

TABLE 2

| | All Recipients | | EGF | | DGF | | p-value |
|---|---|---|---|---|---|---|---|
| Number (N) | 176 | | 106/176 | (60.2%) | 70/176 | (39.8%) | |
| Mean age, | 52.0 | (19-76) | 50.5 | (20-70) | 54.1 | (19-76) | NS |
| years (range) | | | | | | | |
| Gender | | | | | | | |
| Female | 66 | (37.5%) | 45 | (42.5%) | 21 | (29.6%) | NS |
| Male | 110 | (62.5%) | 61 | (57.5%) | 49 | (69.4%) | |
| TX number | | | | | | | |
| First TX | 161 | (91.5%) | 99 | (93.4%) | 62 | (88.6%) | NS |
| Re-TX | 15 | (8.5%) | 7 | (6.6%) | 8 | (11.4%) | |
| Underlying kidney | | | | | | | |
| disease | | | | | | | |
| Polycystic disease | 42 | (23.8%) | 26 | (24.5%) | 16 | (22.9%) | NS |
| Glomerulonephritis | 35 | (19.9%) | 21 | (19.8%) | 14 | (20.0%) | |
| Diabetes Mellitus | 48 | (27.3%) | 29 | (27.4%) | 19 | (27.1%) | |
| Other | 51 | (30.0%) | 30 | (28.3%) | 21 | (30.0%) | |
| Mode of dialysis | | | | | | | |
| Hemodialysis | 119 | (67.6%) | 64 | (60.4%) | 55 | (78.6%) | 0.014 |
| Peritoneal dialysis | 57 | (32.4%) | 42 | (39.6%) | 15 | (21.4%) | |
| Mean time on dialysis, | 850 | (73-4263) | 770 | (73-4263) | 975 | (187-3361) | 0.007 |
| days (range) | | | | | | | |

Tx = transplant

Kidney transplantation, with the inevitable cold ischemia, results in similar pathophysiological changes in the kidney as found in acute kidney injury (AKI) in general and, therefore, can be and generally is regarded as a model of AKI (Heyman et al., Kidney Int. 77: 9-16 (2010)). The characteristics of the transplantations and the kidney graft function and survival, overall and separately, in kidney transplant recipients with either early graft function (EGF) or delayed graft function (DGF) are shown in Table 3.

TABLE 3

| | All Recipients | | EGF | | DGF | | p-value |
|---|---|---|---|---|---|---|---|
| Donor age | 51.8 | (9-75) | 49.1 | (9-75) | 55.8 | (9-75) | 0.002 |
| Mean CIT, | 21.9 | (12.0-31.15) | 21.3 | (12.0-31.2) | 22.9 | (15.3-30.0) | 0.007 |
| hours (range) | | | | | | | |
| Initial CNI | | | | | | | |
| Tacrolimus | 41 | (23.3%) | 24 | (22.6%) | 17 | (24.3%) | NS |
| Cyclosporine A | 135 | (76.7%) | 82 | (77.4%) | 53 | (75.7%) | NS |
| Mean plasma | | | | | | | |
| creatinine, | | | | | | | |
| μmol/L (range) | | | | | | | |
| Day-1 | 531 | (112-1383) | 445 | (112-1353) | 664 | (274-1383) | <0.0001 |
| Day-3 | 407 | (60-1253) | 250 | (60-805) | 644 | (298-1253) | <0.0001 |
| Day-7 | 270 | (53-925) | 141 | (53-333) | 458 | (119-925) | <0.0001 |
| 3 weeks | 153 | (52-530) | 120 | (52-280) | 206 | (67-530) | <0.0001 |
| 3 month | 124 | (56-525) | 110 | (56-200) | 148 | (59-515) | <0.0001 |
| 1 year | 116 | (43-312) | 109 | (43-312) | 128 | (73-276) | 0.002 |
| Mean eGFR, | | | | | | | |
| mL/min (range) | | | | | | | |
| 3 weeks | 57.4 | (15-148) | 64.2 | (25-148) | 46.4 | (15-99) | <0.001 |
| 3 month | 65.5 | (17-175) | 69.7 | (27-175) | 58.9 | (17-113) | 0.003 |
| 1 year | 72.1 | (21-153) | 74.8 | (21-153) | 67.7 | (29-132) | 0.05 |
| Mean urine output, | | | | | | | |
| mL/24 hours (range) | | | | | | | |
| Day-1 | 1756 | (0-9280) | 2544 | (100-9280) | 574 | (0-2350) | <0.0001 |
| Day-3 | 1729 | (0-5970) | 2406 | (150-5970) | 713 | (0-2880) | <0.0001 |
| Day-7 | 1960 | (0-4630) | 2412 | (1500-4630) | 1274 | (0-3530) | <0.0001 |
| Day-14 | 2339 | (0-4720) | 2661 | (1070-4720) | 1888 | (0-4710) | <0.0001 |
| Rejection | 10 | (5.7%) | 4 | (3.8%) | 6 | (8.6%) | NS |
| Mean time to | 16.8 | (7-49) | 8.7 | (7-11) | 20.8 | (14-49) | NS |
| rejection, | | | | | | | |
| days (range) | | | | | | | |

| | | | | |
|---|---|---|---|---|
| 1-year graft survival | 95.5% | 99.4% | 90.0% | 0.005 |

CIT = cold ischemia time;
CNI = calcineurin inhibitor;
eGFR = estimated glomerular filtration rate

Urine samples for analysis of neutrophil gelatinase-associated lipocalin (NGAL) were collected upon arrival and on days 1, 3, 7 and 14 days after transplantation. The urine samples were centrifuged, and the supernatants were stored at −70° C. The pre-transplantation urinary NGAL (uNGAL) levels represent the levels found in dialysis-dependent patients with chronic kidney disease. The uNGAL levels have been previously shown to be elevated in patients with chronic kidney disease (Bolignano et al., Clin. Am. J. Soc. Nephrol. 4: 337-344 (2009); and Malyszko et al., Ren. Fail. 30: 625-628 (2008)). The pre-transplantation levels were used as a reference for post-transplantation uNGAL levels. The samples were analyzed by a two-step chemiluminescent microparticle immunoassay on a standardized clinical platform (ARCHITECT®, Abbott Laboratories, Abbott Park, Ill.) as previously described (Bennett et al., Clin. J. Am. Soc. Nephrol. 3: 665-673 (2008)). The assay used in the studies described herein corresponds to that marketed outside the United States as the NGAL Assay for the Abbott ARCHITECT® instrument (Abbott Laboratories, Abbott Ireland, Longford, Ireland).

SPSS software, version 16.0 (SPSS Inc., Chicago, Ill.), was used for statistical analyses. All of the analyzed variables were tested for distribution. T-test and ANOVA were used for samples with normal distribution, and Mann-Whitney U and Kruskal-Wallis were used for analyses of samples with skewed distribution. Chi-square and Fisher's exact tests were employed in analyses of contingency tables. A multivariate analysis was used to assess predictors of DGF. A receiver operating characteristic (ROC) analysis was performed to assess the potential of NGAL to predict DGF. The odds ratio (OR) was used to assess the relative measure of effect. A p-value <0.05 was considered to be significant.

The primary outcome variable was the onset of graft function after transplantation. DGF was defined as described by Halloran et al. (Transplantation 46: 223-228 (1988)), i.e., oliguria of less than 1 L/24 hrs for more than two days, plasma creatinine concentration greater than 500 μmol/l throughout the first week, or more than one dialysis session needed in the first week. DGF, as described by Halloran et al., was seen in 70/176 (39.8%) transplantations. The kidneys in transplant recipients with DGF started to function at about 12.0 days (mean; range 3-38 days) after transplantation. In comparison to transplant recipients with EGF, the donors for the transplant recipients with DGF were older, the mean cold ischemia time (CIT) was longer, hemodialysis was more common, and time on dialysis before transplantation was longer. All of these factors are regarded as significant risk factors for DGF (Peeters et al., Curr. Opin. Crit. Care 10: 489-498 (2004)). Also in comparison to transplant recipients with EGF, the mean plasma creatinine was significantly higher and the mean estimated glomerular filtration rate (eGFR) was lower in the transplant recipients with DGF. At one year, the overall patient survival was 98.9% and the overall graft survival was 95.5%. Graft survival was significantly lower in transplant recipients with DGF (90.0%) as compared to transplant recipients with EGF (99.4%, p=0.005).

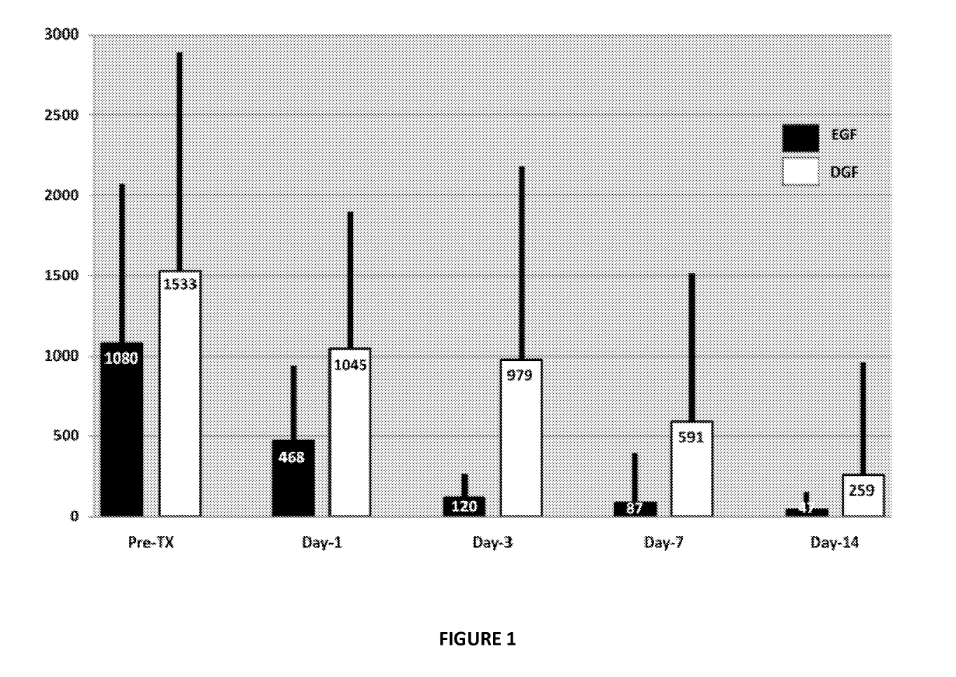

The mean pre-transplantation uNGAL concentration was 1,212 ng/mL (range=85-5,044 ng/mL). The mean pre-transplantation uNGAL concentration in patients with DGF was 1,281 ng/mL (range=250-3,687 ng/mL), whereas the mean pre-transplantation uNGAL concentration in patients with EGF was 1,183 ng/mL (range=85-5,044 ng/mL). The mean uNGAL levels decreased after transplantation in patients with DGF and in patients with EGF. This is shown in FIG. 1, which is a bar graph of time of uNGAL sampling (pre-transplantation (pre-TX), day 1, day 3, day 7 and day 14) vs. concentration of uNGAL in ng/mL in which the white bars represent transplant recipients with DGF and the black bars represent transplant recipients with EGF. Already on day 1, the mean uNGAL concentration was significantly lower in transplant recipients with EGF as compared to transplant recipients with DGF. The difference was statistically significant and persisted at all later time-points.

A ROC analysis was performed to assess the potential of uNGAL in predicting DGF. The area under the curve (AUC) for uNGAL at day 1 was 0.750 (Confidence Interval “CI” 0.663-0.837, p<0.0001). At the optimal cut-off level of 562 ng/mL, the sensitivity was 68% and the specificity was 73%.

In a multivariate analysis, the effect of donor age, donor creatinine, donor eGFR, CIT, recipient age, mod of dialysis before transplantation, time on dialysis before transplantation, urine output on day 1, change in plasma creatinine from pre-transplantation to day 1, and uNGAL of recipient on day 1 at onset of function were assessed. In addition to urine output on day 1 (p<0.0001), only uNGAL on day 1 emerged as a significant predictor of DGF (p=0.0009) as shown in Table 4.

TABLE 4

| | p-value |
|---|---|
| Donor age (years) | 0.550 |
| Donor plasma creatinine (μmol/L) | 0.945 |
| Donor eGFR (mL/min) | 0.890 |
| CIT (hours) | 0.920 |
| Recipient age (years) | 0.551 |
| Mode of dialysis (hemodialysis or peritoneal dialysis) | 0.453 |
| Time on dialysis before TX (days) | 0.886 |
| Change in plasma creatinine from pre-TX to Day-1 | 0.624 |
| Recipient Day-1 urine output (mL) | <0.0001 |
| Recipient Day-1 uNGAL (ng/mL) | 0.0009 |

eGFR = estimated glomerular filtration rate;
CIT = cold ischemia time;
TX = transplant

A ROC analysis was also performed to assess the potential of uNGAL in predicting DGF in those transplant recipients in which, based on early urine output and plasma creatinine, EGF was expected. Initial focus was placed on the 112 transplant recipients having an output of urine on day 1 of greater than 1 liter. Despite good dieresis, 15/112 transplant recipients developed DGF. Their mean uNGAL concentration on day 1 was significantly higher (1,217 ng/mL; range 79-3,600 ng/mL) than the mean uNGAL concentration on day 1 of the 97 transplant recipients with EGF (460 ng/mL; range 16-2,771, p<0.0001). The AUC for uNGAL at day 1 was 0.696 (CI 0.526-0.866, p=0.034). At the cut-off level of 410 ng/mL, the sensitivity was 64% and the specificity was 61%.

Focus was also placed on the 112 transplant recipients in which the plasma concentration of creatinine had decreased from the pre-transplantation level to day 1. Twenty-eight (28) of these transplant recipients had DGF. Their mean uNGAL concentration on day 1 was significantly higher (1,262 ng/mL; range 79-3,600 ng/mL) than the mean uNGAL concentration on day 1 of the 84 transplant recipients with EGF (417 ng/mL, range 16-1,323 ng/mL, p<0.0001). The AUC for uNGAL at day 1 was 0.747 (CI 0.613-0.881, p<0.0001). At the cut-off level of 410 ng/mL, the sensitivity was 71% and the specificity was 58%.

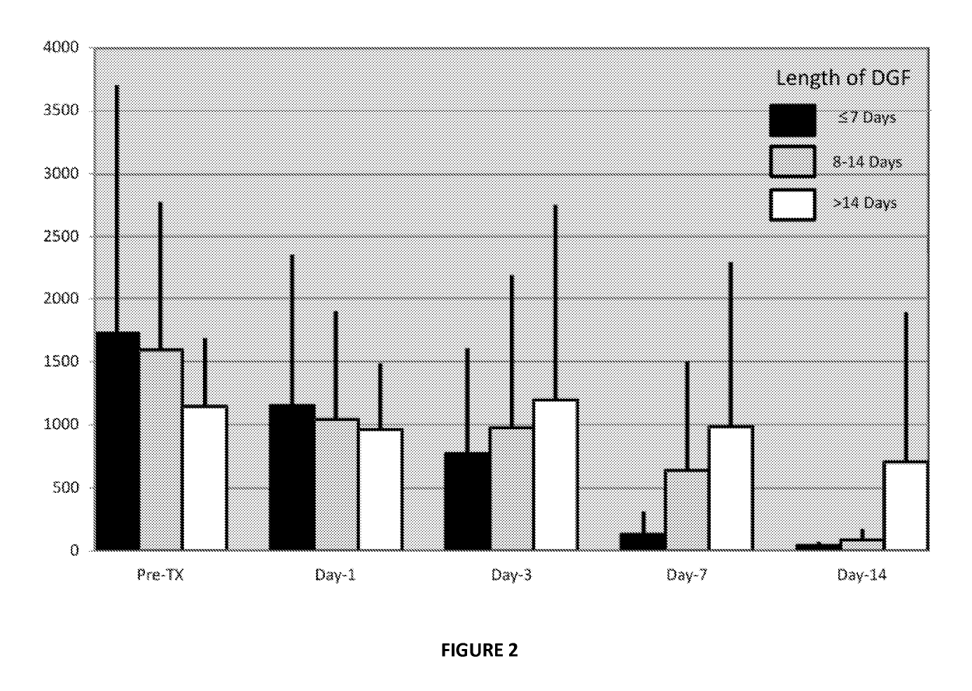

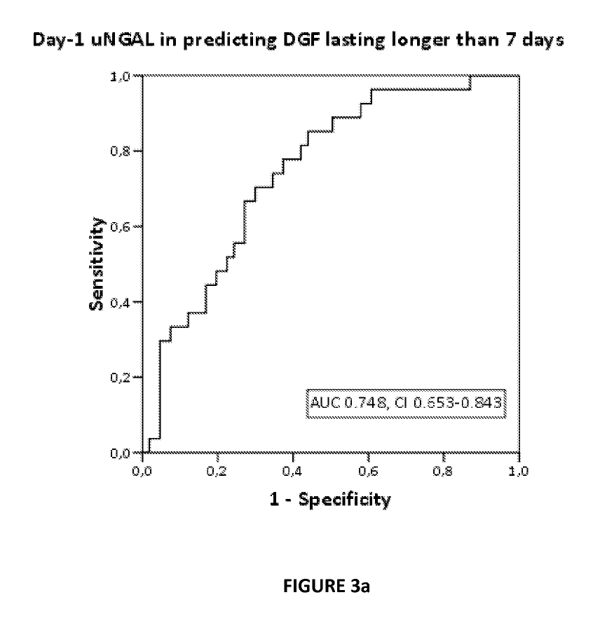

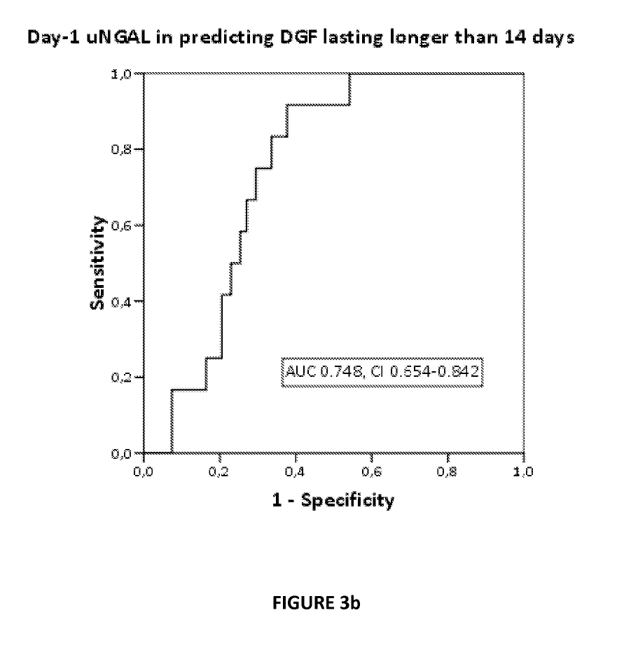

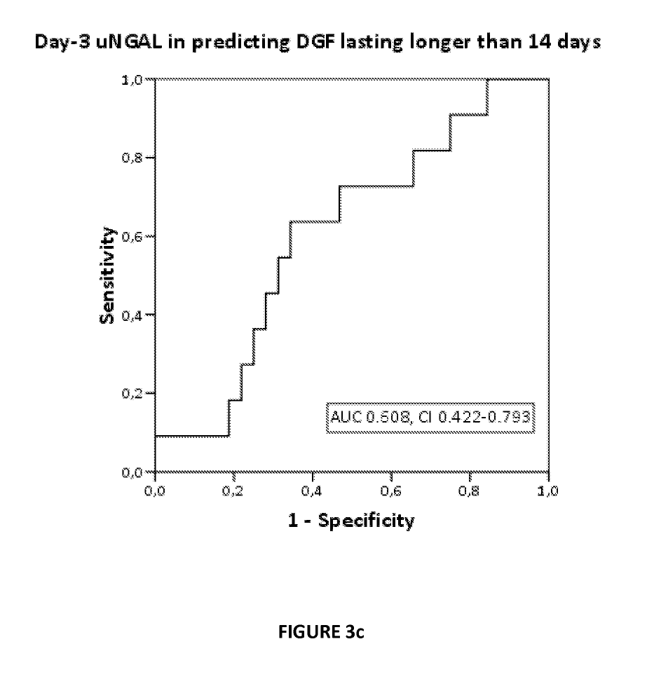

ROC analyses were performed to assess the potential of uNGAL in predicting prolonged DGF. The transplant recipients with DGF (n=70) were divided into three groups according to the duration of the DGF as shown in Table 5 and FIG. 2 (which is a bar graph of time of uNGAL sampling (pre-transplantation (pre-TX), day 1, day 3, day 7 and day 14) vs. concentration of uNGAL in ng/mL in which the white bars represent transplant recipients with DGF for less than or equal to 7 days, the gray bars represent transplant recipients with DGF for 8-14 days, and the black bars represent transplant recipients with DGF for more than 14 days): group 1 had DGF for less than or equal to 7 days (n=17), group 2 had DGF for 8-14 days (n=27), and group 3 had DGF for more than 14 days (n=26). As shown in FIG. 3a (which is a graph of specificity vs. sensitivity for uNGAL concentration on day 1 in predicting DGF lasting longer than 7 days), the ROC analysis for uNGAL concentration on day 1 in predicting DGF lasting longer than 7 days had an AUC of 0.748 (CI 0.653-0.843, p<0.0001). At the optimal cut-off level of 508 ng/mL, the sensitivity was 74% and the specificity was 65%. As shown in FIG. 3b (which is a graph of specificity vs. sensitivity for uNGAL concentration on day 1 in predicting DGF lasting longer than 14 days), the ROC analysis for uNGAL concentration on day 1 in predicting DGF lasting longer than 14 days had an AUC of 0.748 (CI 0.654-0.842, p=0.005). At the optimal cut-off level of 645 ng/mL, the sensitivity was 75% and the specificity was 70%. ROC analysis of uNGAL on day 3 was not significant, as shown in FIG. 3c, which is a graph of specificity vs. sensitivity for uNGAL concentration on day 3 in predicting DGF lasting longer than 14 days.

TABLE 5

| | DGF lasting less | | DGF lasting | | DGF lasting | | |
|---|---|---|---|---|---|---|---|
| | than 7 days | | between 8 and | | more than 14 | | p- |
| | (n = 17) | | 14 days (n = 27) | | days (n = 26) | | value |
| Mean donor age, | 55.2 | (35-67) | 56.0 | (9-75) | 56.0 | (26-67) | NS |
| years (range) | | | | | | | |
| Mean CIT, hours | 23.4 | (16.9-30.0) | 22.0 | (15.3-29.0) | 23.3 | (15.6-29.4) | NS |
| (range) | | | | | | | |
| Recipient age, | 54.0 | (36-66) | 56.2 | (19-73) | 52.0 | (21-77) | NS |
| years (range) | | | | | | | |
| Mode of dialysis | | | | | | | |
| before TX | | | | | | | |
| Hemodialysis | 15 | | 21 | | 19 | | NS |
| Peritoneal dialysis | 2 | | 6 | | 7 | | NS |
| Mean time on dialysis | 1082 | (469-3239) | 939 | (207-3361) | 929 | (187-9211) | NS |
| before TX, days | | | | | | | |
| (range) | | | | | | | |
| Mean Day-1 urine | 755 | (0-2090) | 519 | (0-2350) | 439 | (0-1840) | NS |
| output, mL (range) | | | | | | | |
| Mean uNGAL, ng/mL | | | | | | | |
| (range) | | | | | | | |
| Pre-TX | 1733 | (250-5960) | 1597 | (405-3687) | 1150 | (726-1938) | NS |
| Day-1 | 1153 | (223-3600) | 1044 | (79-2634) | 963 | (364-2090) | NS |
| Day-3 | 774 | (133-2568) | 982 | (85-4946) | 1200 | (173-5686) | NS |
| Day-7 | 133 | (19-730) | 642 | (29-3749) | 984 | (71-4759) | 0.001 |
| Day-14 | 39 | (6-108) | 84 | (4-379) | 706 | (19-4550) | 0.004 |
| Mean plasma | 115 | (73-196) | 117 | (73-211) | 156 | (90-276) | 0.002 |
| creatinine at 1 year | | | | | | | |
| post-TX, μmol/L | | | | | | | |
| (range) | | | | | | | |
| Mean eGFR | 75.5 | (37-127) | 70.7 | (37-132) | 56.4 | (29-93) | 0.031 |
| at 1 year post-TX, | | | | | | | |
| mL/min (range) | | | | | | | |

| | | | | |
|---|---|---|---|---|
| 1 year graft survival | 100% | 100% | 73.1% | 0.001 |

CIT = cold ischemia time;
TX = transplant

Using the conventional definition of Humar et al. (Clin. Transplant 11: 623-627 (1997)), i.e., the need for dialysis during the first week after transplantation, DGF was found in 66 transplant recipients, slow graft function (SGF; plasma creatinine remained above 265 μmol/l for more than five days after transplantation but without need for dialysis) was found in 16 of the remaining 110 transplant recipients, and immediate graft function (IGF) was found in 94 transplant recipients. Urinary NGAL concentrations were significantly higher in the DGF group (according to definition of Humar et al.) as compared to the SGF group (according to definition of Humar et al.) and the IGF group (according to definition of Humar et al.) as shown in Table 6.

TABLE 6

| | IGF (n = 94) | | SGF (n = 16) | | DGF (n = 66) | | p-value |
|---|---|---|---|---|---|---|---|
| Mean uNGAL, | | | | | | | |
| ng/mL (range) | | | | | | | |
| Pre-TX | 1002 | (25-5044) | 1858 | (405-5960) | 1429 | (250-3687) | NS |
| Day-1 | 476 | (18-2771) | 704 | (42-2433) | 887 | (79-2634) | <0.0001 |
| Day-3 | 82 | (79-2634)^{a} | 288 | (86-730)^{a,c} | 588 | (59-2349)^{c} | <0.0001 |
| Day-7 | 55 | (6-498)^{b} | 128 | (20-730)^{b,d} | 218 | (19-821)^{d} | <0.0001 |
| Day-14 | 34 | (2-306) | 34 | (9-64)^{e} | 59 | (6-287)^{e} | 0.011 |
| Mean plasma | | | | | | | |
| creatinine, μmol/L | | | | | | | |
| (range) | | | | | | | |
| Day-1 | 425 | (112-916)^{a} | 710 | (429-1353)^{a} | 640 | (274-1383) | <0.0001 |
| Day-3 | 215 | (60-585)^{a} | 653 | (409-1044)^{a} | 620 | (194-1253) | <0.0001 |
| Day-7 | 135 | (53-523)^{a} | 304 | (177-628)^{a,d} | 450 | (119-925)^{d} | <0.0001 |
| 3 weeks | 116 | (52-280)^{c} | 152 | (109-297)^{c} | 209 | (67-530) | <0.0001 |
| 3 months | 107 | 856-183)^{c} | 128 | (84-200)^{c} | 149 | (59-515) | <0.0001 |
| 1 year | 107 | (43-312) | 118 | (57-252) | 130 | (71-276) | 0.002 |
| Mean eGFR, | | | | | | | |
| mL/min (range) | | | | | | | |
| 3 weeks | 66.1 | (34-148) | 55.7 | (25-82) | 44.5 | (15-99) | <0.0001 |
| 3 months | 71 | (31-175) | 66 | (27-113) | 57 | (17-106) | 0.001 |
| 1 year | 75.7 | (21-153) | 75.9 | (22-127) | 65.5 | (29-132) | 0.03 |

| | | | | |
|---|---|---|---|---|
| 1 year graft | 98.9% | 100% | 89.4% | 0.012 |
| survival | | | | |

Groups defined according to Humar et al. ((1997), supra):
^{a}p < 0.0001 between the IGF and SGF group,
^{b}p = 0.01 between the IGF and SGF groups,
^{c}p ≦ 0.005 between the DGF and SGF groups,
^{d}p = 0.009 between the DGF and SGF groups, and
^{e}p = 0.031 between the DGF and SGF groups.
GFR = estimated glomerular filtration rate

Early after transplantation the NGAL levels in the SGF group were similar to the NGAL levels in the DGF group. At later time-points, the NGAL levels in the SGF group were similar to the NGAL levels in the IGF group. One year after transplantation, the mean plasma creatinine was higher and the eGFR was lower in the DGF group as compared to the IGF group and the SGF group (ANOVA: p=0.002 and 0.03, respectively). A ROC analysis was performed to assess the potential of uNGAL in predicting conventionally defined DGF. The AUC was 0.736 (CI 0.642-0.830, p<0.0001). At the optimal cut-off level of 514 ng/mL, the sensitivity was 69% and the specificity was 69%. There were no significant differences in mean uNGAL concentrations between the transplant recipients with DGF as defined by Halloran (Halloran et al. (1988), supra) and conventionally defined DGF (Humar et al. (1997), supra).

All transplant recipients with EGF were divided into “high” and “low” groups based on uNGAL concentration at days 1, 3, 7 and 14 using the medians 346 ng/mL, 64 ng/mL, 31 ng/mL, and 17 ng/mL, respectively, as cut-off points. High uNGAL concentrations at day 1 were associated with higher plasma creatinine and lower eGFR levels at three weeks post-transplantation but not at later time-points. High uNGAL concentrations at day 3 were associated with a mean eGFR of 59.1 ml/min (range 25-147) at three weeks post-transplantation, whereas low uNGAL concentrations at day 3 were associated with a mean eGFR of 70.9 ml/min (range 38-112, p=0.04) at three weeks post-transplantation.

In summary, uNGAL levels decreased after transplantation in recipients with DGF and EGF. The reduction in uNGAL was significantly greater in transplant recipients with EGF at all measured time-points. It was found that the level of uNGAL at day 1 could differentiate those transplant recipients with DGF from those transplant recipients with EGF, even in transplant recipients with decreasing plasma creatinine and copious dieresis. Urinary NGAL concentration at day 1 predicted DGF with an AUC of 0.750, a sensitivity of 68%, and a specificity of 73%. Urinary NGAL concentration at day 1 also predicted DGF lasting longer than 14 days with an AUC of 0.748, a sensitivity of 75%, and a specificity of 70%. A uNGAL level above the median in transplant recipients with EGF was associated with worse kidney function at three weeks after transplantation but not at later time-points. Thus, uNGAL concentration at day 1 predicts DGF and prolonged DGF with moderate sensitivity and specificity and in those transplant recipients with clinical signs of EGF. High uNGAL in transplant recipients with clinical signs of EGF was associated with worse kidney function at three weeks after transplantation but was not predictive of kidney function at later time-points.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. In this regard, where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, it should be understood that, although the present invention has been specifically disclosed in the context of preferred embodiments and optional features, those skilled in the art may resort to modifications and variations of the concepts disclosed herein. Such modifications and variations are considered to be within the scope of the invention as defined by the appended claims.

What is claimed is:

1. A method of prognosticating cadaveric kidney transplant function in a patient, which method comprises:
assaying urine samples from the patient for urinary neutrophil gelatinase-associated lipocalin (uNGAL) to allow detection of a change in the level of uNGAL in a urine sample from the patient at about 24 hours after cadaveric kidney transplantation in comparison to the level of uNGAL in a urine sample from the patient at within about 24 hours before cadaveric kidney transplantation, wherein assaying comprises detecting the level of uNGAL in the urine sample, wherein:
detecting a decrease in the level of uNGAL of less than or equal to about 55% in the level of uNGAL in a urine sample from the patient at about 24 hours after cadaveric kidney transplantation in comparison to the level of uNGAL in a urine sample from the patient at within about 24 hours before cadaveric kidney transplantation predicts delayed graft function (DGF), whereas detecting a decrease in the level of uNGAL of greater than about 55% in a urine sample from the patient at about 24 hours after cadaveric kidney transplantation in comparison to the level of uNGAL in a urine sample from the patient at within about 24 hours before cadaveric kidney transplantation predicts early graft function (EGF),
whereupon cadaveric kidney transplant function in a patient is prognosticated.

2. The method of claim 1, which further comprises determining the volume of urine voided by the patient within about 24 hours of transplantation, wherein a volume less than about one liter indicates DGF and a volume greater than about one liter indicates EGF.

3. The method of claim 1, wherein detecting a decrease in the level of uNGAL of less than or equal to about 20% in a urine sample from the patient at about 24 hours after cadaveric kidney transplantation in comparison to the level of uNGAL in a urine sample from the patient at within about 24 hours before cadaveric kidney transplantation predicts DGF lasting more than about 14 days.

4. The method of claim 3, which further comprises:
assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation, wherein assaying comprises detecting the level of uNGAL in the urine sample, wherein:
detecting an increase in the level of uNGAL in a urine sample from the patient at about 72 hours after cadaveric kidney transplantation in comparison to the level of uNGAL in a urine sample from the patient at about 24 hours after cadaveric kidney transplantation further predicts DGF lasting more than about 14 days.

5. The method of claim 1, wherein detecting a decrease in the level of uNGAL of at least about 20% and less than about 35% in a urine sample from the patient at about 24 hours after cadaveric kidney transplantation in comparison to the level of uNGAL in a urine sample from the patient at within about 24 hours before cadaveric kidney transplantation predicts DGF lasting less than about 14 days.

6. The method of claim 5, which further comprises:
assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation, wherein assaying comprises detecting the level of uNGAL in the urine sample,
wherein:
detecting a decrease in the level of uNGAL of at least about 30% in a urine sample from the patient at about 72 hours after cadaveric kidney transplantation in comparison to the level of uNGAL in a urine sample from the patient at about 24 hours after cadaveric kidney transplantation predicts DGF lasting≦7 days,
and detecting a decrease in the level of uNGAL of less than about 10% in a urine sample from the patient at about 72 hours after cadaveric kidney transplantation in comparison to the level of uNGAL in a urine sample from the patient at about 24 hours after cadaveric kidney transplantation predicts DGF lasting from about 8 days to about 14 days.

7. A method of prognosticating cadaveric kidney transplant function in a patient diagnosed with DGF, which method comprises:
assaying urine samples from the patient for uNGAL to allow detection of a change in the level of uNGAL in a urine sample from the patient at about 72 hours after cadaveric kidney transplantation in comparison to the level of uNGAL in a urine sample from the patient at about 24 hours after cadaveric kidney transplantation, wherein assaying comprises detecting the level of uNGAL in the urine sample,
wherein:
detecting a decrease in the level of uNGAL of at least about 30% in a urine sample from the patient at about 72 hours after cadaveric kidney transplantation in comparison to the level of uNGAL in a urine sample from the patient at about 24 hours after cadaveric kidney transplantation predicts DGF lasting≦7 days,
detecting a decrease in the level of uNGAL of less than about 10% in a urine sample from the patient at about 72 hours after cadaveric kidney transplantation in comparison to the level of uNGAL in a urine sample from the patient at about 24 hours after cadaveric kidney transplantation predicts DGF lasting from about 8 days to about 14 days, or
detecting an increase in the level of uNGAL in a urine sample from the patient at about 72 hours after cadaveric kidney transplantation in comparison to the level of uNGAL in a urine sample from the patient at about 24 hours after cadaveric kidney transplantation predicts DGF lasting longer than about 14 days,
whereupon kidney transplant function in a patient diagnosed with DGF is prognosticated.

8. A method of assessing risk of DGF in a patient, who has been diagnosed with EGF based on (i) the volume of urine voided by the patient within about 24 hours of cadaveric kidney transplantation being at least about one liter and/or (ii) the level of creatinine in the plasma of the patient at about 24 hours after cadaveric kidney transplantation being lower than the level of creatinine in the plasma of the patient within about 24 hours before cadaveric kidney transplantation, which method comprises:
assaying urine samples from the patient for uNGAL to allow detection of a change in the level of uNGAL in a urine sample from the patient at about 24 hours after cadaveric kidney transplantation in comparison to the level of uNGAL in a urine sample from the patient within about 24 hours before cadaveric kidney transplantation, wherein assaying comprises detecting the level of uNGAL in the urine sample and:
detecting a decrease in the level of uNGAL of less than about 55% in a urine sample from the patient at about 24 hours after cadaveric kidney transplantation in comparison to the level of uNGAL in a urine sample from the patient within about 24 hours before cadaveric kidney transplantation predicts risk of DGF,
whereupon risk of DGF in a patient diagnosed with EGF is assessed.

9. A kit comprising at least one component for assaying urine from a patient, who has received a cadaveric kidney transplant, has received a cadaveric kidney transplant and has been diagnosed with EGF, or is about to receive a cadaveric kidney transplant, for neutrophil gelatinase-associated lipocalin (NGAL) and instructions for assaying the urine for NGAL and assessing kidney transplant function or risk of DGF in the patient.
